(19) United States
(12) Patent Application Publication
(10) Pub. No.: US 2005/0008625 A1
(43) Pub. Date: Jan. 13, 2005

(54) Antibody affinity engineering by serial epitope-guided complementarity replacement

(75) Inventors: Robert F. Balint, Palo Alto, CA (US); Jeng-Horng Her, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KaloBios, Inc., Mountain View, CA (US)

(21) Appl. No.: 10/778,726

(22) Filed: Feb. 13, 2004

Related U.S. Application Data
(60) Provisional application No. 60/447,846, filed on Feb. 13, 2003.

(51) Int. Cl. A61K 48/00

(52) U.S. Cl. 424/93.21; 514/44

(57) ABSTRACT

This invention provides for a novel means for obtaining human idiologs for any non-human antibody to any target by epitope guided replacement of variable regions using competitive cell-based methods in which the competitor can be either the reference antibody or a ligand that binds to the same epitope on the target as the reference antibody.

### CROSS-REFERENCES TO RELATED APPLICATIONS

This application claims the benefit of U.S. Provisional Patent Application No. 60/447,846, filed on Feb. 13, 2003, the teachings of which are herein incorporated by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Many therapeutic targets suitable for antibody-mediated therapy have been validated with the use of non-human antibody reagents, and this process is expected to continue for the many new therapeutic targets for antibodies which are expected to emerge from the human genome in the coming years. As a target becomes validated for therapy, the antibodies, typically murine, used to validate the target become leads for the development of biologic drugs. However, for many therapeutic applications the efficacy and safety of non-human antibodies are severely compromised by their tendency to induce strong immune responses in patients, as a result of which the antibodies are eliminated and lose efficacy. In cases where very large doses must be used, there is also a risk of systemic anaphylactic response with unacceptably toxic consequences. Thus, before such antibodies can be approved for therapeutic use they must be replaced with human counterparts having equivalent bioactivity, or they must be “humanized” in some way to eliminate or minimize immunogenicity in humans. Existing methods for the isolation of human antibodies with required bioactivities for therapeutic use, or for “humanizing” non-human antibodies for therapeutic use, have many limitations which are overcome by the systems and methods of the present invention.

Established methods for the isolation of antigen-specific human antibodies include the screening of hybridomas (Kohler & Milstein, 1975, Nature 256:495-497) from mice which transgenic for the human immunoglobulin loci (refs), and the in vitro display methods, which include phage display (e.g., McCafferty et al., 1990, Nature 348:552-554), yeast display (e.g., Boder and Wittrup, 1997, Nat Biotechnol 15:553-557), and ribosome display (e.g., Hanes and Pluckthun, 1997, Proc Natl Acad Sci U S A 94:4937-4942). These methods have yielded many useful human antibodies, however, they allow little control over epitope selection, making the isolation of antibodies with bioactivities which are equivalent to those of non-human therapeutic lead antibodies a highly uncertain enterprise.

Hybridoma methods rely on the humoral immune responses of mice, and are therefore subject to the uncertainties of such responses, which may include epitope biases and failure to respond well to antigens which are homologous to host proteins. Epitope biases may arbitrarily exclude antibodies from selection which bind to certain epitopes on the native antigen surface, which do not elicit useful responses in mice. Such excluded epitopes often include epitopes which are required for desired bioactivities. In fact, selection in mice may often be biased against desired bioactivities precisely because such bioactivities may interfere with the immune response of the animal. Furthermore, mice transgenic for human immunoglobulin loci generally do not express the full complement of human diversity, and therefore the success rates for desired affinities and specificities tend to be even lower than with conventional mice. Hybridoma methods are also hampered by time and labor demands. Available screening methods are highly inefficient, requiring many man-hours to screen even modest numbers of clones for desired specificities and affinities.

The principal limitations of the display technologies stem from the requirement for antigen stability in vitro, and from the lack of robust methods for affinity maturation. For selection of antibodies in vitro, antigens must be purified and immobilized on artificial surfaces, and they must remain stable in this state typically for many hours before bound antibodies can be recovered from them. Most proteins undergo varying degrees of denaturation when attached to foreign materials, and this produces strong epitope biases. Because the kinetics of binding to immobilized antigens are slow, prolonged periods of exposure are required, and this too increases the risk of denaturation. When antigens and antibodies denature, non-specific binding may occur, and if denaturation occurs after binding, binding may become irreversible and bona-fide binders may thus be lost.

Chain-guided selection has been used with phage display in an attempt to bias human antibody selection toward epitopes bound by non-human lead antibodies (Hoogenboom ref and patent). This strategy has met with only limited success, however, due to the great difficulty of finding a suitable heavy chain using a light chain guide. Light chain V-regions which have been affinity matured in vivo typically have little direct affinity for the antigen, but rather have been extensively optimized for stabilization of the complex with their high-affinity heavy chain partners. Thus, heavy chains which fortuitously coordinate with an affinity-matured light chain from another species to bind the same epitope in the same orientation with comparable affinity must be exceedingly rare in human heavy chain repertoire libraries. The problem is compounded by the fact that phage display libraries are heavily biased toward antibody chains which express well in bacteria, which in fact comprise only a small minority of natural human antibody chains. Thus, even if suitable heavy chain partners for the guiding light chains exist in the repertoire they are likely to be lost in the process of generating the phage-displayed library.

The most widely used methods for minimizing the immunogenicity of non-human antibodies while retaining as much of the original specificity and affinity as possible involve grafting the CDRs of the non-human antibody onto human germline frameworks selected for their structural homology to the non-human framework (Jones P T, Dear P H, Foote J, Neuberger M S, Winter G. Replacing the complementarity-determining regions in a human antibody with those from a mouse. Nature. 1986 321:522-5.). Typically this results in a substantial loss of affinity, at least some of which can be regained by making mutational adjustments in the structure based on sophisticated modeling techniques combined with trial and error. Often recovery of affinity requires retaining some residues from the non-human parent. In spite of this, however, in many cases the full affinity of the original non-human antibody has not been recovered. Exemplary methods for humanization of antibodies are disclosed in, for example, U.S. Pat. No. 6,180,370.

### BRIEF SUMMARY OF THE INVENTION

This invention provides for a novel means to generate improved antibodies. The general approach is designated antibody engineering by Serial Epitope-Guided Complementarity Replacement or SECR™.

The invention relates to new antibody compositions, interaction systems incorporating these novel antibody compositions, and methods of using these interaction systems and the novel antibody compositions. In one embodiment, the novel antibody compositions of the invention comprise antibodies in complexes with other components of an interaction assay. In other embodiments, the novel antibody compositions comprise hybrid antibodies that have one antibody chain from a reference antibody and a second antibody chain from a different source. The antibodies of the invention can be in the form of Fab, scFv, Fv, dab, Fd fragments, single heavy chain v-region domains (VH), single light chain v-region domains (VL), light chain-light chain antibody, heavy chain-heavy-chain antibody, and single light chains or heavy chains. In one embodiment, the compositions of the invention comprise a plurality of antibodies individually complexed to a component of an interaction system. In a preferred embodiment, the plurality of antibodies is a library of antibodies having a desired level of diversity.

The interaction systems of the invention can be used to replace the antigen-binding variable region domains (V-regions) of a reference antibody with unrelated V-regions that bind to the same antigen. In a preferred embodiment the replacement antibody binds to the same epitope as the reference antibody. Antibodies specific for an antigen are isolated from the libraries of the invention by using the antigen as a “bait” molecule in one of the interaction systems of the invention.

A non-human reference antibody can be rendered non-immunogenic in humans using the interaction systems of the invention in a process in which the V-regions of the reference antibody are replaced one at a time with V-regions from diverse libraries of human V-regions while retaining the original binding specificity of the reference antibody. This strategy, termed Serial Epitope-guided Complementarity Replacement (SECR), is enabled by setting up a competition in cells between a “competitor” and a library of diverse hybrids of the reference antibody (“test antibodies”) for binding to limiting amounts of antigen in the presence of a reporter system which responds to the binding of test antibody to antigen. In one embodiment the competitor is the reference antibody or derivative thereof such as a single-chain Fv fragment. In another embodiment the competitor is a natural or artificial ligand of the antigen which binds to the same epitope as the reference antibody. Artificial ligands might include scaffolded peptides selected from random peptide libraries displayed on the surface of a protein scaffold such as thioredoxin. The only requirements of the competitor are that it bind the same epitope as the reference antibody, and that it compete with the reference antibody for antigen binding in one of the interaction systems of the invention. In all embodiments the test antibodies are hybrids of the reference antibody having one antigen-binding V-region in common from the reference antibody, and the other V-region is selected at random from a diverse source such as a repertoire library from non-immune donors. The common V-region from the reference antibody serves as a guide, positioning the test antibodies on the same epitope on the antigen, and in the same orientation, so that selection is biased toward the highest antigen-binding fidelity to the reference antibody.

Many types of reporter system may be used in the invention to detect desired interactions between test antibodies and antigen. For example, complementing reporter fragments, such as those disclosed in U.S. patent application Ser. No. 09/526,106 may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Similarly, the reporter-inhibitor fusion and the reactivator of an auto-inhibited reporter reactivation system or RAIR, such as those disclosed in U.S. patent application Ser. No. 10/208,730, may be fused to antigen and test antibody and used in the same way to select antigen-binding antibodies in the presence of competitors. Alternatively, the reporter and inhibitor of a competitive activation system COMPAC™, such as those disclosed in U.S. patent application Ser. No. 10/076,845, may be linked to antigen and competitor, so that the reporter is inhibited by antigen-competitor binding, and activated in the presence of any test antibody that competes with the competitor for antigen binding.

In a SECR™ selection each cell expresses a single test antibody along with the competitor, antigen, and reporter components, so that each test antibody competes one-on-one with the competitor for binding to limiting antigen, and the activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. In a preferred embodiment at least one hybrid antibody selected in the first round will have an affinity for the antigen comparable to or higher than that of the reference antibody.

In the second V-region replacement step, the human V-regions selected in the first step become the guides for the selection of human replacements for the remaining non-human reference antibody V-region from a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. In a preferred embodiment at least one of the selected human antibodies binds to the same epitope on the same antigen as the reference antibody. In a more preferred embodiment, at least one of the selected human antibodies binds to the same epitope with an affinity which is comparable to or higher than that of the reference antibody. In a most preferred embodiment, at least one of the resulting human idiologs is comparable or superior in bioactivity to the reference antibody.

### BRIEF DESCRIPTION OF THE DRAWINGS

FIG. 1. Illustration of the process of epitope-guided replacement of target-specific antibody V-regions using a cell-based competitive activation system. Three components are expressed in each cell: (1) target antigen linked to reporter, (2) competitor linked to inhibitor, and (3) antibody library member comprised of a reference V-region and a member of a library of companion V-regions. When target and competitor bind to each other the reporter is inhibited and the selectable phenotype is extinguished. Antibodies in the library which can compete with the competitor for binding to the target activate the reporter, and can thus be identified by the selectable phenotype conferred by the active reporter on the cells. The presence of the reference V-region in the library helps guide the selection to precisely the same epitope as that bound by the reference antibody itself.

FIG. 2. In the second step of the process of epitope-guided replacement of target-specific antibody V-regions using a cell-based competitive activation system, the second V-region is replaced using the first selected V-region as a guide. In this step the hybrid antibody resulting from the first round of selection is used as the competitor, with the inhibitor linked to the reference chain in the case of bi-molecular antibody formats such as Fab. The result is a fully human idiolog of the reference antibody with identical binding specificity and comparable or better affinity.

FIG. 3. Conversion of a chimeric Fab fragment to a fully human idiolog with comparable or better affinity for the same epitope by serial epitope-guided complementarity replacement (SECR). The target epitope and the V-regions of the mouse antibody are used to guide the selection of new human V-regions from large libraries, with comparable or higher affinities than those of the originals, but with the same orientation on the epitope for maximum retention of bioactivity. This is accomplished by deploying the murine antibody as a chimeric Fab with its antigen in the β-lactamase competitive selection system as described above for selection of high-affinity VL domains in Fabs guided by antigen-binding VH domains. In this case the chimeric Fab serves as both competitor and reference Fab. Typically, murine Fabs can be chimerized with little loss in affinity by fusing the mouse V-regions to homologous human constant regions. The chimeric Fab may then be expressed with the β-lactainase inhibitor, BLIP, fused via flexible linker to the amino terminus of either VH or VL, depending on which V-region is to be replaced first. As before, the antigen is fused to either terminus of β-lactamase, so that binding of the chimeric Fab to the antigen docks BLIP to β-lactamase, inactivating the latter. New Fabs are then selected for their ability to compete with the chimeric Fab for antigen binding, and thereby activate β-lactamase by competitively displacing the inhibitor. As shown in FIG. 3, it is usually preferable to replace the VL domain first, so that the dominant affinity of the mouse VH domain can be exploited to retain the original orientation of the antibody on the epitope while guiding the selection of a high-affinity human VL domain.

FIG. 4. In this figure, the high-affinity VL domain can then provide a strong orientation guide for selection of a high-affinity human VH domain. In this way, by preserving not just the epitope, but the orientation of the antibody on the epitope as well, the full bioactivity of the original antibody should be faithfully preserved. The β-lactamase structure is rendered from the x-ray coordinates of Jelsch et al., 1993 (Proteins Struct. Funct. 16, 364ff) using the WebLab ViewerPro 3.7 program (Molecular Simulations, Inc., San Diego). The BLIP structure and the BLIP-β-lactamase complex were rendered from the x-ray coordinates of Lim et al., 2001, Nat.Struct.Biol. 8:848ff; and Strynadka et al., 1996, Nat.Struct.Biol. 3:290ff.

FIG. 5. Vectors for conversion of chimeric Fab fragments to fully human idiologs with comparable or better affinity for the same epitope by serial epitope-guided chain replacement (SECR). A. The antigen—β-lactamase fusion and BLIP-competitor fusion are expressed from variants of the lactose operon promoter (lacp; clacp) in a p15A-based vector (Rose, 1988, Nucleic Acids Res. 16:355). The preferred competitor is a fragment of the reference antibody such as an Fab in which the non-human constant regions have been replaced by human constant regions (chimeric Fd and chimeric LC). The BLIP-Fab fusion is expressed from a dicistronic cassette comprised of signal peptide (SP) for secretion to the periplasmic space, and internal ribosome entry site (IRES) for translation of the second cistron. When VL is to be replaced first, the reference chimeric Fd is expressed from the upstream cistron to insure its excess over the reference chimeric LC, and BLIP is fused to the LC. Test antibodies are expressed from a variant of lacp in a pBR322-based vector, and are comprised of a SP, V-region derived from the reference antibody and a library of diverse companion V-regions. In Fab format, the test antibodies are expressed from a dicistronic cassette identical to that of the BLIP-competitor fusion. B. Vectors for the second step, i.e., VH selection. The human VL selected in the first step becomes the guide for VH selection and the semi-chimeric Fab containing the selected human VL becomes the competitor fused to BLIP via the LC. The test molecules are fully human, being comprised of the human VL guide and a library of diverse human VH domains. BLIP^{D49A} is a low-affinity mutant of BLIP; (G₄S)₃, flexible hydrophilic 15-mer linker comprised of (Gly₄Ser)₃; tt, transcription terminator; kan, kanamycin resistance gene; ori, origin of replication; fl ori, filamentous phage origin of replication for transformation by phage transfection; cat, chloramphenicol resistance gene.

FIG. 6. Structural illustration of the formation of the complex of β-lactamase and BLIP as mediated by the interaction of an antibody Fab fragment with the extra-cellular domain of human CD40 antigen. The β-lactamase structure is rendered from the x-ray coordinates of Jelsch et al., 1993 (Proteins Struct. Funct. 16, 364ff) using the WebLab ViewerPro 3.7 program (Molecular Simulations, Inc., San Diego). The BLIP structure and the BLIP—β-lactamase complex were rendered from the x-ray coordinates of Lim et al., 2001, Nat.Struct.Biol. 8:848ff; and Strynadka et al., 1996, Nat.Struct.Biol. 3:290ff. The Fab structure was rendered from the x-ray coordinates of a model Fab (Jeffrey et al., 1995 Nature Struct Biol 2:466ff), and the human CD40 extra-cellular domain was rendered from the coordinates of Bajorath and Aruffo, 1997, Proteins: Struct, Funct., Genet. 27:59-70.

FIG. 7. Vectors for selection of high-affinity human VH domains for the HB08 chimeric anti-human CD40 Fab. The variable region coding sequences of HB08 were recovered from a hybridoma cell line by PCR with primers specific for the VH and VL framework 1 and framework 4 regions, respectively (Larrick and Balint, 1994, in Antibody Techniques, Malik and Lillehoj, Eds., Academic Press, New York, pp. 103-114). These sequences were then subcloned into the β-lactamase inhibition and competitive activator vectors shown in FIG. 5 for expression as Fab fragments with human CH1 and CL constant regions. The Fabs in the two vectors were expressed from dicistronic transcripts with the common chain as the upstream cistron to ensure that it is in excess over the competing chains. A. The coding sequence of the human CD40 extra-cellular domain (CD40ED; Bajorath and Aruffo, 1997, Proteins: Struct, Funct, Genet 27:59-70) was inserted into the β-lactamase inhibition vector for expression of CD40 fused to the amino terminus of β-lactamase, while the HB08 chimeric Fab was expressed from the same vector with BLIP fused to the amino terminus of the light chain for light chain selection. To establish optimum selection conditions the parent HB08 chimeric Fab was also expressed as the test antibody from the competitive activator vector, so that the same Fab competed with itself for inhibition or activation, of β-lactamase. The coding sequence of the original mouse VL domain in the light chain on the competitive activator vector was then replaced with a human non-immune repertoire of ˜10⁷ human VL domains. B. Vectors for the second step, i.e., selection of human VH companion for VL1B3 selected in the first round. VL1B3 becomes the guide for VH selection, and the semi-chimeric Fab containing HB08 VH and VL1B3 becomes the competitor fused to BLIP via the LC. The test molecules are fully human, being comprised of the VL1B3 guide and a non-immune repertoire library of ˜10⁸ human VH domains. See description of FIG. 5 for abbreviations.

FIG. 8. Graphs of plating efficiencies of E. coli cells expressing CD40—β-lactamase fusion, BLIP fused to Fab fragment of HB08 anti-CD40 murine antibody, and free HB08 Fab (positive control) or anti-GST Fab (negative control). Approximately 10,000 cells were seeded on each of the indicated concentrations of ampicillin in solid 2×YT medium containing 10 μM IPTG to induce transcription, and grown overnight at 33° C. Plating efficiencies were determined as the percentage of cells plated forming colonies, and plotted on a semi-log scale.

### DETAILED DESCRIPTION OF THE INVENTION

### Definitions

“Affinity matured” in the context of antibodies refers to an antibody that is derived from a reference antibody, binds to the same antigen as the reference antibody, and has a higher affinity for that antigen than that of the reference antibody. In a preferred embodiment, the affinity matured antibody binds to the same epitope as the reference antibody.

As used herein, an “antibody” refers to a protein functionally defined as a binding protein and structurally defined as comprising an amino acid sequence that is recognized by one of skill as being derived from the framework region of an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer, is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to g3p (see, e.g., U.S. Pat. No: 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies include all those that have been displayed on phage (e.g., scFv, Fv, Fab and disulfide linked Fv (Reiter et al. (1995) Protein Eng. 8: 1323-1331). Antibodies can also include diantibodies and miniantibodies.

“Antigen” refers to substances which are capable, under appropriate conditions, of inducing a specific immune response and of reacting with the products of that response, that is, with specific antibodies or specifically sensitized T-lymphocytes, or both. Antigens may be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” may be used to refer to any substance to which an antibody binds, or for which antibodies are desired, regardless of whether the substance is immunogenic. For such antigens, antibodies may be identified by recombinant methods, independently of any immune response.

“Antibody library” refers to a repertoire or synthetic library of genes encoding antibodies or antibody fragments such as Fab, scFv, Fd, LC, VH, or VL, which is obtained from the natural ensemble, or “repertoire”, of antibodies present in human donors, and obtained primarily from the cells of peripheral blood and spleen. In a preferred embodiment, the human donors are “non-immune”, i.e., not presenting with symptoms of infection.

“Binding” refers to the adherence of molecules to one another, for example, enzymes to substrates, antibodies to antigens, DNA strands to their complementary strands. Binding occurs because the shape and chemical natures of parts of the molecules surfaces are complementary. A common metaphor is the “lock-and-key,” used to describe how enzymes fit around their substrate.

A “binding ensemble member” refers to a molecule that participates in a specific binding interaction with another member of the binding ensemble. A binding ensemble often comprises two members, i.e., a binding pair, but can comprise three or more members. For example, an antigen and two antibodies that recognize two different epitopes on an antigen and can be bound to the antigen at the same time are members of a binding ensemble. Binding ensemble members can include, for example, antibodies/antigens, receptor/ligands, biotin/avidin, and interacting protein domains such as leucine zippers and the like. A binding ensemble member as used herein can be a binding domain, i.e., a subsequence of a protein that binds specifically to another member of the binding ensemble. In reference to binding pairs, the binding pair members can also be referred to as a binding pair member and a binding partner (or cognate binding partner). Binding ensembles can also include members that are added to dock other binding ensemble members to the responder/reporter complex or the reactivator complex, such as, for example, biotin/avidin, antibody/antigen, or any receptor/ligand.

“Chimeric polynucleotide” means that the polynucleotide comprises regions which are wild-type and regions which are mutated. It may also mean that the polynucleotide comprises wild-type regions from one polynucleotide and wild-type regions from another related polynucleotide.

“Competitor” refers to that molecule whose binding to the antigen or target molecule in the systems of the invention leads to inhibition of the reporter. Thus, in the systems of the invention, test molecules must compete with the competitor for binding to the antigen or target molecule in order to activate the reporter and be selected.

“Complementarity-determining region” and “CDR” refer to the art-recognized term as exemplified by the Kabat and Chothia CDR definitions also generally known as hypervariable regions or hypervariable loops (Chothia and Lesk (1987) J. Mol. Biol. 196: 901; Chothia et al. (1989) Nature 342: 877; E. A. Kabat et al., Sequences of Proteins of Immunological Interest (National Institutes of Health, Bethesda, Md.) (1987); and Tramontano et al. (1990) J. Mol. Biol. 215: 175). Variable region domains typically comprise the amino-terminal approximately 105-115 amino acids of a naturally-occurring immunoglobulin chain (e.g., amino acids 1-110), although variable domains somewhat shorter or longer are also suitable for forming single-chain antibodies.

“Epitope” refers to that portion of an antigen or other macromolecule capable of forming a binding interaction that interacts with the variable region binding pocket of an antibody. Typically, such binding interaction is manifested as an intermolecular contact with one or more amino acid residues of a CDR.

“Expression vector” includes vectors which are capable of expressing nucleic acid sequences contained therein, i.e., any nucleic acid sequence which is capable of effecting expression of a specified nucleic acid code disposed therein (the coding sequences are operably linked to other sequences capable of effecting their expression). Some expression vectors are replicable in the host organism either as episomes or as an integral part of the chromosomal DNA. A useful, but not a necessary, element of an effective expression vector is a marker encoding sequence—i.e. a sequence encoding a protein which results in a phenotypic property (e.g. tetracycline resistance) of the cells containing the protein which permits those cells to be readily identified. Expression vectors are frequently in the form of plasmids or viruses. However, the invention is intended to include such other forms of expression vectors which serve equivalent functions and which may, from time to time become known in the art.

“Guide Chain” refers to a portion of a reference antibody comprising one of the variable region domains (V-region), which is incorporated into the test antibodies as a guide to the epitope bound by the reference antibody. Test antibodies can be selected in a variety of formats, but the Fab fragment format and the single-chain Fv (scFv) fragment format are preferred. In the Fab format the guide chain is either the Fd chain or the light chain, and its V-region is obtained from the reference antibody. The V-region of the other chain will be supplied from a diverse pool of cognate V-regions comprising a natural repertoire, a synthetic repertoire, or a combination of both. In the scFv format, the guide “chain” is a reference antibody V-region genetically linked to a diverse pool of cognate V-regions comprising a natural repertoire, a synthetic repertoire, or a combination of both. In either case the guide chain generally constrains the binding of the test antibodies to the same epitope on the antigen bound by the reference antibody, and preferably in the same orientation.

“Homologs” means polypeptides having the same or conserved residues at a corresponding position in their primary, secondary or tertiary structure. The term also extends to two or more nucleotide sequences encoding the homologous polypeptides. Example homologous peptides are the immunoglobulin isotypes.

“Host cell” refers to a prokaryotic or eukaryotic cell into which the vectors of the invention may be introduced, expressed and/or propagated. Typical prokaryotic host cells include various strains of E. coli. Typical eukaryotic host cells are yeast or filamentous fungi, or mammalian cells, such as Chinese hamster ovary cells, murine NIH 3t3 fibroblasts, or human embryonic kidney 193 cells.

“Idiolog” is an antibody which shares its antigen and epitope specificity with another antibody, e.g., a reference antibody, but which is not derived from that antibody.

An “inhibitor” refers to a molecule that can inhibit the responder/reporter when both are in the same complex. The complex may be a responder complex as described in the reactivation systems of U.S. Provisional Patent Application No. 60/373,802, or the reporter complex formed in competitive activation systems such as described in U.S. patent application Ser. No. 10/076,845 when the inhibitor and reporter are brought together by the binding ensemble.

“Interaction systems” means any system that generates a detectable signal from a responder/reporter in such a way that it can be generically used to study the interaction of binding ensembles. Preferred embodiments are complementation systems typically fragment complementation, auto-reactivation systems (RAIR™), and/or competitive activation (COMPAC™) systems.

“Isolated” refers to a nucleic acid or polypeptide separated not only from other nucleic acids or polypeptides that are present in the natural source of the nucleic acid or polypeptide, but also from polypeptides, and preferably refers to a nucleic acid or polypeptide found in the presence of (if anything) only a solvent, buffer, ion, or other component normally present in a solution of the same. The terms “isolated” and “purified” do not encompass nucleic acids or polypeptides present in their natural source.

“Library” means a collection of nucleotides sequences, e.g., DNA, encoding antibodies within clones; or a genetically diverse collection of antibody polypeptides.

“Link” or “join” or “fuse” refers to any method of functionally connecting peptides, typically covalently, including, without limitation, recombinant fusion of the coding sequences, and covalent bonding (e.g., disulfide bonding). In the systems of the invention, a binding pair member is typically linked or joined or fused, often using recombinant techniques, at the amino-terminus or carboxyl-terminus by a peptide bond to a responder or to an activator or inhibitor of the responder. However, the binding pair member may also be inserted into the responder or inhibitor at an internal location that can accept such insertions. The binding pair member can either directly adjoin the fragment to which it is linked or fused or it can be indirectly linked or fused, e.g., via a linker sequence. “Linked” may also refer to a non-covalent physical association, particularly one which is constitutive, i.e., does not require docking, under operating conditions. For example, in a responder mixture comprised of responder, inhibitor, and binding ensemble member, each component is typically linked to at least one other component, either covalently, e.g., via peptide linkage, or non-covalently, via high-affinity binding interaction.

“Phenotype” refers to a physical (e.g., pigment, or cell shape) and/or metabolic property of a cell which can be measured or exploited in some fashion and which is effected by the reporter gene.

“Purified” means that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

“Recombinant nucleic acid” refers to a nucleic acid in a form not normally found in nature. That is, a recombinant nucleic acid is flanked by a nucleotide sequence not naturally flanking the nucleic acid or has a sequence not normally found in nature. Recombinant nucleic acids can be originally formed in vitro by the manipulation of nucleic acid by restriction endonucleases, or alternatively using such techniques as polymerase chain reaction. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention.

“Recombinant polypeptide” refers to a polypeptide expressed from a recombinant nucleic acid, or a polypeptide that is chemically synthesized in vitro.

“Recombinant variant” refers to any polypeptide differing from naturally occurring polypeptides by amino acid insertions, deletions, and substitutions, created using recombinant DNA techniques. Guidance in determining which amino acid residues may be replaced, added, or deleted without abolishing activities of interest, such as enzymatic or binding activities, may be found by comparing the sequence of the particular polypeptide with that of homologous peptides and minimizing the number of amino acid sequence changes made in regions of high homology.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

“Reference antibody” refers to an antibody for which the practitioner wants to obtain a variant with “improved” characteristics. In a preferred embodiment, the improved characteristic is affinity for the same antigen. In another preferred embodiment, the improved characteristic is reduced immunogenicity, obtained by substituting human chains for the non-human chains of the reference antibody.

The term “responder” refers to any protein that produces a detectable signal, including, but not limited to detectable signals such as fluorescence, enzymatic activity, a selectable phenotype (e.g., antibiotic resistance), a screenable phenotype, or that produces an activity that results in a phenotypic change or provides a functional product.

The term “reporter” refers specifically to a responder that produces a detectable signal, or that confers a selectable phenotype, i.e., a responder whose sole purpose is to signal the occurance of an event, rather than to use the event as a trigger for some other action.

The term “responder complex” refers to a complex comprised of the responder, the inhibitor, and a binding ensemble member. One or more members of the responder complex may be complexed with the others by a binding interaction. At least one of member of the responder complex may also be in the same polypeptide chain as at least one other member. In a preferred embodiment, all three members of the responder complex are in the same polypeptide chain.

“Synthetic antibody library” refers to a library of genes encoding one or more antibodies or antibody fragments such as Fab, scFv, Fd, LC, VH, or VL, in which one or more of the complementarity-determining regions (CDR) has been partially or fully randomized by oligonucleotide-directed mutagenesis. “Randomized” means that part or all of the sequence encoding the CDR has been replaced by sequence randomly encoding all twenty amino acids or some subset of the amino acids.

“Target” may be used to refer to the molecule to which a reference antibody binds, “reference antibody” being an antibody for which the practitioner wants to obtain a variant with “improved” characteristics. Thus, “target” may herein be used synonymously with “antigen”.

“Test antibody” refers to a variant of the reference antibody which is typically a member of a library of such variants which in the invention is subjected to conditions wherein any member which binds to the target is selected. In a preferred embodiment, test antibodies comprise one V-region from the reference antibody and the other V-region from a diverse population which is not derived from the reference antibody, but which comprises a natural or synthetic repertoire of antibody V-regions.

“Vector” refers to a plasmid or phage or virus or vector, for expressing a polypeptide from a DNA (RNA) sequence. The vector can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate translation initiation and termination sequences. Structural units intended for use in yeast or eukaryotic expression systems may include a leader sequence enabling extracellular secretion of translated protein by a host cell.

Each of the above terms is meant to encompass all that is described, unless the context dictates otherwise.

### Introduction

The systems and methods of the present invention overcome many limitations of current methods for the isolation of antibodies, particularly human antibodies, which bind to antigens of choice with high affinities and specificities. Human antibodies are required for many therapeutic applications. However, current methods for obtaining human antibodies with required bioactivities for therapeutic use are often unreliable. These methods include isolating antigen-specific hybridomas from human antibody-producing transgenic mice, and isolating antigen-specific human antibody genes from libraries displayed on bacteriophage, cells, or ribosomes by biopanning.

Hybridoma technologies rely on the humoral immune responses of mice, and are therefore subject to the uncertainties of such responses, which may include epitope biases and failure to respond well to antigens which are homologous to host proteins. Epitope biases may arbitrarily exclude antibodies from selection which bind to certain epitopes on the native antigen surface, which do not elicit useful responses in mice. Such excluded epitopes often include epitopes which are required for desired bioactivities. In fact, selection in mice may often be biased against desired bioactivities precisely because such bioactivities may interfere with the immune response of the animal. Hybridoma technologies are also hampered by time and labor demands. Available screening methods are highly inefficient, requiring many man-hours to screen even modest numbers of clones for desired specificities and affinities, compared to the numbers of clones that can be screened by in vitro methods. Also, mice transgenic for human immunoglobulin loci generally do not express the full complement of human diversity, and therefore the success rates for desired affinities and specificities tend to be lower than with conventional mice. It will be shown that the systems and methods of the present invention allow precise control over epitope specificity, and enable thorough searching of complete human antibody repertoires.

The principal limitations of the display technologies stem from the requirement for antigen stability in vitro, and from the lack of robust methods for affinity maturation. For selection of antibodies in vitro, antigens must be purified and immobilized on artificial surfaces, and they must remain stable in this state typically for many hours before bound antibodies can be recovered from them. Most proteins undergo varying degrees of denaturation when attached to foreign material, and this produces strong epitope biases. Because the kinetics of binding to immobilized antigens are slow, prolonged periods of exposure are required, and this too increases the risk of denaturation. When antigens and antibodies denature, non-specific binding may occur, and if denaturation occurs after binding, binding may become irreversible and bona-fide binders may thus be lost.

Chain-guided selection has been used with phage display in an attempt to bias human antibody selection toward epitopes bound by non-human lead antibodies (Hoogenboom ref and patent). This strategy has met with only limited success, however, due to the great difficulty of finding a suitable heavy chain using a light chain guide. Light chain V-regions which have been affinity matured in vivo typically have little direct affinity for the antigen, but rather have been extensively optimized for stabilization of the complex with their high-affinity heavy chain partners. Thus, heavy chains which fortuitously coordinate with an affinity-matured light chain from another species to bind the same epitope in the same orientation with comparable affinity must be exceedingly rare in human heavy chain repertoire libraries. The problem is compounded by the fact that phage display libraries are heavily biased toward antibody chains which express well in bacteria, which in fact comprise only a small minority of natural human antibody chains. Thus, even if suitable heavy chain partners for the guiding light chains exist in the repertoire they are likely to be lost in the process of generating the phage-displayed library. It will be shown that the systems and methods of the present invention overcome many of the limitations of in vitro display methods by allowing access to a greater proportion of the natural diversity of human repertoires.

The most widely used methods for minimizing the immunogenicity of non-human antibodies while retaining as much of the original specificity and affinity as possible involve grafting the CDRs of the non-human antibody onto human germline frameworks selected for their structural homology to the non-human framework (Jones P T, Dear P H, Foote J, Neuberger M S, Winter G. Replacing the complementarity-determining regions in a human antibody with those from a mouse. Nature. 1986 321:522-5).

Typically this results in a substantial loss of affinity, at least some of which can be regained by making mutational adjustments in the structure based on sophisticated modeling techniques combined with trial and error. Often recovery of affinity requires retaining some residues from the non-human parent. In spite of this, however, in many cases the full affinity of the original non-human antibody has not been recovered. Thus, “humanization” often results in antibodies which retain some of the immunogenicity of the non-human parent, whereas, the systems and methods of the present invention enable the selection of fully human antibodies with comparable bioactivity to the non-human parent.

### Nucleic Acids and Polypeptides

Antibodies are polypeptides comprising at least a heavy chain variable region and a light chain variable region that together specifically bind and recognize an antigen, the variable regions being specified by immunoglobulin genes. Recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chain variable regions respectively.

Antibodies exist, e.g., as intact immunoglobulins, as a number of well-characterized fragments produced by digestion with various peptidases, or as well-characterized fragments produced by recombinant gene expression. Thus, for example, pepsin digests an antibody below the disulfide linkages in the hinge region to produce F(ab)′2, a dimer of Fab which itself is a light chain joined to VH-CH1 (Fd fragment) by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

Often the polypeptides of the invention are produced by recombinant expression of nucleic acids encoding the polypeptides. Expression methodology is well known to those of skill in the art. Such a recombinant polypeptide can be made by ligating the appropriate nucleic acid sequences encoding the desired amino acid sequences by methods known in the art, in the proper reading frame, and expressing the product by methods known in the art (see, e.g., Scopes, Protein Purification. Principles and Practice, Springer-Verlag, New York 1994; Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997).

Nucleic acids encoding the polypeptides of the invention can be obtained using routine techniques in the field of recombinant genetics (see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997).

Often, the nucleic acid sequences encoding the polypeptides of the invention are cloned from cDNA and genomic DNA libraries by hybridization with probes, or isolated using amplification techniques with oligonucleotide primers. Amplification techniques can be used to amplify and isolate sequences from DNA or RNA (see, e.g., Dieffenbach & Dveksler, PCR Primers: A Laboratory Manual (1995)). Alternatively, overlapping oligonucleotides can be produced synthetically and joined to produce one or more of the domains. Nucleic acids encoding the component domains can also be isolated from expression libraries using antibodies as probes.

In an example of obtaining a nucleic acid encoding a polypeptide of the invention using PCR, the nucleic acid sequence or subsequence is PCR amplified, using a sense primer containing one restriction site and an antisense primer containing another restriction site. This will produce a nucleic acid encoding the desired polypeptide and having terminal restriction sites. This nucleic acid can then be easily ligated into a vector having the appropriate corresponding restriction sites. If the desired polypeptide is a fusion protein, the domains can be directly joined or may be separated by a linker, or other, protein sequence. Suitable PCR primers can be determined by one of skill in the art using the sequence information provided in GenBank or other sources. Appropriate restriction sites can also be added to the nucleic acid encoding the protein or protein subsequence by site-directed mutagenesis. The plasmid containing the polypeptide encoding sequence of the invention is cleaved with the appropriate restriction endonuclease and then ligated into an appropriate vector for amplification and/or expression according to standard methods.

Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

In some embodiments, it may be desirable to modify the polypeptides of the invention. One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

In some embodiments, the recombinant nucleic acids encoding the polypeptides of the invention are modified to provide preferred codons which enhance translation of the nucleic acid in a selected organism (e.g., yeast preferred codons are substituted into a coding nucleic acid for expression in yeast).

### Expression Cassettes and host Cells for Expressing Polypeptides of the Invention

There are many expression systems for producing the polypeptides of the invention that are well know to those of ordinary skill in the art. (See, e.g., Gene Expression Systems, Fernandes and Hoeffler, Eds. Academic Press, 1999.) Typically, the polynucleotide that encodes the polypeptide is placed under the control of a promoter that is functional in the desired host cell. An extremely wide variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites, enhancers, operators, and the like are also optionally included. Constructs that include one or more of these control sequences are termed “expression cassettes.” Accordingly, the nucleic acids that encode the joined polypeptides are incorporated for the desired level of expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25); and the lambda-derived P_{L} promoter and N-gene ribosome binding site (Shimatake et al., Nature (1981) 292: 128). The particular promoter system is not critical to the invention, any available promoter that functions in prokaryotes can be used. Standard bacterial expression vectors include plasmids such as pBR322-based plasmids, e.g., pBLUESCRIPT™, pSKF, pET23D, λ-phage derived vectors, p15A-based vectors (Rose, Nucleic Acids Res. (1988) 16:355 and 356) and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

For expression of fusion polypeptides in prokaryotic cells other than E. coli, regulatory sequences for transcription and translation that function in the particular prokaryotic species are required. Such promoters can be obtained from genes that have been cloned from the species, or heterologous promoters can be used. For example, the hybrid trp-lac promoter functions in Bacillus in addition to E. coli. These and other suitable bacterial promoters are well known in the art and are described, e.g., in Sambrook et al. and Ausubel et al. Bacterial expression systems for expressing the proteins of the invention are available in, e.g., E. coli, Bacillus sp., and Salmonella (Palva et al., Gene 22:229-235 (1983); Mosbach et al., Nature 302:543-545 (1983). Kits for such expression systems are commercially available.

Similarly, for expression of the polypeptides of the invention in eukaryotic cells, transcription and translation sequences that function in the particular eukaryotic species are required. For example, eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Either constitutive or regulated promoters can be used in the present invention. Regulated promoters can be advantageous because the concentration of heterologous protein in the host cell can be controlled. An inducible promoter is a promoter that directs expression of a gene where the level of expression is alterable by environmental or developmental factors such as, for example, temperature, pH, anaerobic or aerobic conditions, light, transcription factors and chemicals.

For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l. Acad Sci. USA 82: 1074-8). These promoters and their use are discussed in Sambrook et al., supra.

Inducible promoters for other organisms are also well known to those of skill in the art. These include, for example, the metallothionein promoter, the heat shock promoter, as well as many others.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The construction of polynucleotide constructs generally requires the use of vectors able to replicate in host bacterial cells, or able to integrate into the genome of host bacterial cells. Such vectors are commonly used in the art. A plethora of kits are commercially available for the purification of plasmids from bacteria (for example, EasyPrepJ, FlexiPrepJ, from Pharmacia Biotech; StrataCleanJ, from Stratagene; and, QIAexpress Expression System, Qiagen). The isolated and purified plasmids can then be further manipulated to produce other plasmids, and used to transform cells.

The polypeptides of the invention can be expressed intracellularly, or can be secreted from the cell. Intracellular expression often results in high yields. If necessary, the amount of soluble, active polypeptide may be increased by performing refolding procedures (see, e.g., Sambrook et al., supra.; Marston et al., Bio/Technology (1984) 2: 800; Schoner et al., Bio/Technology (1985) 3: 151). Polypeptides of the invention can be expressed in a variety of host cells, including E. coli, other bacterial hosts, yeast, and various higher eukaryotic cells such as the COS, CHO and HeLa cells lines and myeloma cell lines. The host cells can be mammalian cells, insect cells, or microorganisms, such as, for example, yeast cells, bacterial cells, or fungal cells.

Once expressed, the recombinant polypeptides can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to 95% homogeneity are preferred, and 98 to 99% or more homogeneity are most preferred.

To facilitate purification of the polypeptides of the invention, the nucleic acids that encode the polypeptides can also include a coding sequence for an epitope or “tag” for which an affinity binding reagent is available. Examples of suitable epitopes include the myc and V-responder genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

One of skill would recognize that modifications can be made to the protein domains without diminishing their biological activity. Some modifications may be made to facilitate the cloning, expression, or incorporation of a domain into a polypeptide. Such modifications are well known to those of skill in the art and include, for example, the addition of codons at either terminus of the polynucleotide that encodes the binding domain to provide, for example, a methionine added at the amino terminus to provide an initiation site, or additional amino acids (e.g., poly His) placed on either terminus to create conveniently located restriction sites or termination codons or purification sequences.

### Generation of antibody Diversity

Antibody diversity refers primarily to the diversity of antibody binding sites in a group or library of antibody-encoding genes. The specificity of an antibody's binding site is determined by its CDRs (complementarity determining regions). Light chains and/or heavy chains of an antibody have 3 CDRs (CDR1, CDR2, and CDR3). An immunoglobulin light chain or heavy chain variable region consists of a “framework” region interrupted by the three CDRs (hypervariable regions). The extent of the framework region and CDRs have been precisely defined (see, “Sequences of Proteins of Immunological Interest,” E. Kabat et al., 4th Ed., U.S. Department of Health and Human Services, Bethesda, Md. (1987)). The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of an antigen.

The diversity of binding sites for a group or library of antibodies is thus a reflection of the diversity of sequence in the CDRs, the degree of combinatorial combinations of these CDRs in the light and heavy chains, and the degree of combinatorial combinations of the light and/or heavy chains in a group or library of antibodies. All three of these variables may be manipulated to generate a group or library of antibodies having a desired level of diversity. Methods for generating antibody binding diversity are disclosed, for example, in U.S. Pat. No. 6,096,551.

For example, the diversity of an antibody library can be increased by shuffling the heavy and light chain genes (Kang et al., Proc. Nat'l Acad. Sci. 88:11120-11123 (1991)), or by altering the CDRs of the cloned antibody genes (Barbas et al., Proc. Nat'l Acad Sci. 89:4457-4461 (1992); Gram et al., Proc. Nat'l Acad. Sci. 89:3576-3580 (1992)).

Mutagenesis of the CDRs can be done by error-prone replication, replicative incorporation of degenerate oligonucleotides, or by the methods of chemical or UV mutagenesis. These methods are well-known in the art and are disclosed, for example, in Sambrook et al. and Ausubel et al.

PCR may also be used to create amino acid sequence variants of the polynucleotides of the invention. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide or protein at the position specified by the primer. The product DNA fragments replace the corresponding region in the plasmid and this gives the desired amino acid variant. In another embodiment, error prone PCR may be used to generate amino acid variants in the polynucleotides that encode the antibodies.

In another method, polynucleotides encoding the antibodies of the invention are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives the desired amino acid variant.

A further technique for generating amino acid variants is the cassette mutagenesis technique described in Wells et al., Gene 34:315 (1985); and other mutagenesis techniques well known in the art, such as, for example, the techniques in Sambrook et al., supra, and Current Protocols in Molecular Biology, Ausubel et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences are capable of hybridizing to the appropriate novel nucleic acid sequence under stringent conditions.

Shuffling of the CDRs within an antibody polypeptide chain may be accomplished by techniques which are well known in the art. U.S. Pat. Nos. 6,096,551 and 6,372,497 teach exemplary methods for shuffling antibody CDRs.

The light chains and heavy chains, light chains and light chains, or heavy chains and heavy chains may be combinatorial combined to produce the desired level of binding diversity within the antibody library. U.S. Pat. Nos. 5,885,793, 6,300,064, and 6,096,551 teach exemplary methods for “shuffling” antibody chains to increase diversity.

Parsimonious mutagenesis as disclosed in Balint et al. Gene 137(1):109-18 (1993), is a method for maximizing binding diversity in an antibody library with a minimal number of changes within the CDRs. This method creates libraries with low-redundancy ‘doping’ codons and biased nucleotide mixtures designed to maximize the abundance of combining sites with predetermined proportions of preselected sets of alternative amino acids. This allows the library to ‘probe’ the surface of the antigen one or a few amino acid residues at a time with a wide selection of amino acid side chains to search out and identify new high-affinity contacts.

### Antibodies

Structurally, the simplest antibody (IgG) comprises four polypeptide chains, two heavy (H) chains and two light (L) chains inter-connected by disulphide bonds. In some mammals, the light chains exist in two distinct forms called kappa (K) and lambda (λ). Each chain has a constant region (C) and a variable region (V). Each chain is organized into a series of domains. The light chains have two domains, corresponding to the C region and the other to the V region. The heavy chains have four domains, one corresponding to the V region and three domains (1,2 and 3) in the C region. The antibody has two arms (each arm being a Fab region), each of which has a VL and a VH region associated with each other. It is this pair of V regions (VL and VH) that differ from one antibody to another (owing to amino acid sequence variations), and which together are responsible for recognizing the antigen and providing an antigen binding site. In even more detail, each V region is made up from three complementarity determining regions (CDR) separated by four framework regions (FR). The CDRs are the most variable part of the variable regions, and they perform the critical antigen binding function. The CDR regions are derived from many potential germ line sequences via a complex process involving recombination, mutation and selection.

It has been shown that the function of binding antigens can be performed by fragments of a whole antibody. Exemplary binding fragments are (i) the Fab fragment consisting of the VL, VH, CL and CH1 domains; (ii) the Fd fragment consisting of the VH and CH1 domains; (iii) the Fv fragment consisting of the VL and VH domains of a single arm of an antibody, (iv) the dAb fragment (Ward, E. S. et al., Nature 341, 544-546 (1989) which consists of a VH domain; (v) isolated CDR regions; and (vi) F(ab′).sub.2 fragments, a bivalent fragment comprising two Fab fragments linked by a disulphide bridge at the hinge region.

Although the two domains of the Fv fragment are encoded in separate genes, it has proved possible to make a synthetic linker that enables them to be made as a single protein chain (known as single chain Fv (scFv); Bird, R. E. et al., Science 242, 423-426 (1988) Huston, J. S. et al., Proc. Natl. Acad. Sci., USA 85, 5879-5883 (1988)) by recombinant methods. These scFv fragments were assembled from genes from monoclonals that had been previously isolated. In this application, the applicants describe a process to assemble scFv fragments from VH and VL domains that are not part of an antibody that has been previously isolated.

Monoclonal Antibodies

Owing to their high specificity for a given antigen, the advent of monoclonal antibodies (Kohler, G. and Milstein C; 1975 Nature 256: 495) represented a significant technical break-through with important consequences both scientifically and commercially.

Monoclonal antibodies are traditionally made by establishing an immortal mammalian cell line which is derived from a single immunoglobulin producing cell secreting one form of a biologically functional antibody molecule with a particular specificity. Because the antibody-secreting mammalian cell line is immortal, the characteristics of the antibody are reproducible from batch to batch. The key properties of monoclonal antibodies are their specificity for a particular antigen and the reproducibility with which they can be manufactured.

Early methods for producing monoclonals were laborious and time consuming. An animal of choice, e.g., a mouse, was immunized with a desired antigen, antibody producing cells were harvested from the animal (usually by splenectomy) and fused to a suitable immortalized cells, e.g., myeloma cells to make a hybridoma that clonally produces an antibody. Such hybridoma technology is disclosed, for example, in U.S. Pat. Nos. 4,172,124 and 4,196,265; Zurawski et al, Federation Proceedings 39:4922 (1980); Frankel and Gerhard, Molecular Immunology, 16:101-106 (1979).

The introduction of transgenic animals that produce fully human antibodies has permitted the selection of hybridomas which also produce fully human antibodies. Such transgeneic animals are disclosed, for example, in U.S. Pat. Nos. 6,075,181 and 6, 300,129.

Display technologies have also permitted the selection of monoclonal antibodies that are fully human or other animal, chimeric, synthetic, and/or semi-synthetic. Examples of such display technologies are phage display (examples are disclosed in U.S. Pat. Nos. 5,821,047, 5,922,545, 5,403,484, 5,885,793, and 6,291,650) or yeast display (examples are disclosed in U.S. Pat. No. 6,300,065).

Chimeric Antibodies and Synthetic Antibodies

The early monoclonal technologies described above produced non-human antibodies. These antibodies are potentially immunogenic in humans and this immunogenicity has severely hampered the development of therapeutic antibodies. The production of so called “chimeric antibodies,” e.g., variable regions from one species joined to constant regions from another species, has been somewhat successful, but does not overcome the immunogenicity problem in many cases. Exemplary methods for chimerizing antibodies are disclosed in, for example, U.S. Pat. No. 4,816,567.

Recombinant DNA technology has been utilized to produce immunoglobulins which have human framework regions from one species combined with complementarity determining regions (CDR's) from a another species' immunoglobulin (see, e.g., EPO Publication No. 0239400). These new proteins are called “reshaped” or “humanized” (when the framework regions are human) immunoglobulins and the process by which the donor immunoglobulin is converted into a human-like immunoglobulin by combining its CDR's with a human framework is called “humanization”. Exemplary methods for humanization of antibodies are disclosed in, for example, U.S. Pat. No. 6,180,370.

Artificial antibodies and fragments thereof can be constructed based on known antibody sequences, which reflect the structural properties of a whole group of homologous antibody genes. Therefore it is possible to reduce the number of different genes without any loss in the structural repertoire. This approach leads to a limited set of artificial genes, which can be synthesized de novo, thereby allowing introduction of cleavage sites and removing unwanted cleavages sites. Furthermore, this approach enables (i), adapting the codon usage of the genes to that of highly expressed genes in any desired host cell and (ii), analyzing all possible pairs of antibody light (L) and heavy (H) chains in terms of interaction preference, antigen preference or recombinant expression titer, which is virtually impossible using the complete collection of antibody genes of an organism and all combinations thereof.

The use of a limited set of completely synthetic genes makes it possible to create cleavage sites at the boundaries of encoded structural sub-elements. Therefore, each gene is built up from modules which represent structural sub-elements on the protein/(poly)peptide level. In the case of antibodies, the modules consist of “framework” and “CDR” modules. By creating separate framework and CDR modules, different combinatorial assembly possibilities are enabled. Moreover, if two or more artificial genes carry identical pairs of cleavage sites at the boundaries of each of the genetic sub-elements, pre-built libraries of sub-elements can be inserted in these genes simultaneously, without any additional information related to any particular gene sequence. This strategy enables rapid optimization of, for example, antibody affinity, since DNA cassettes encoding libraries of genetic sub-elements can be (i), pre-built, stored and reused and (ii), inserted in any of these sequences at the right position without knowing the actual sequence or having to determine the sequence of the individual library member. Exemplary methods for generating synthetic libraries of antibodies are disclosed in, for example, U.S. Pat. No. 5,885,793 and 6,300,064.

Antibody Libraries

Naïve Libraries and Immunized Libraries.

Naïve libraries are made from the B-lymphocytes of a suitable host which has not been challenged with any immunogen, nor which is exhibiting symptoms of infection or inflammation. Immunized libraries are made a from a mixture of B-cells and plasma cells obtained from a suitably “immunized” host, i.e., a host that has been challenged with an immunogen. In one embodiment, the mRNA from these cells is translated into cDNA using methods well known in the art (e.g., oligo-dT primers and reverse transcriptase). In an alternative embodiment, nucleic acids encoding antibodies from the host cells (mRNA or genomic DNA) are amplified by PCR with suitable primers. Primers for such antibody gene amplifications are well known in the art (e.g., U.S. Pat. No. 6,096,551 and PCT Patent Application WO 00/70023A1 disclose such primers). In a hybrid embodiment, the mRNA from the host cells is synthesized into cDNA and these cDNAs are then amplified in a PCR reaction with antibody specific primers (e.g., U.S. Pat. No. 6,319,690 discloses such a hybrid method). Alternatively, the repertoires may be cloned by conventional cDNA cloning technology (Sambrook and Russell, eds, Molecular Cloning: A Laboratory Manual, 3rd Ed, vols. 1-3, Cold Spring Harbor Laboratory Press, 2001), without using PCR.

Synthetic Libraries.

In one embodiment, a database of published antibody sequences of human origin is established where the antibody sequences are aligned to each other. The database is used to define subgroups of antibody sequences which show a high degree of similarity in both the sequence and the canonical fold of CDR loops (as determined by analysis of antibody structures). For each of the subgroups a consensus sequence is deduced which represents the members of this subgroup; the complete collection of consensus sequences represent therefore the complete structural repertoire of human antibodies.

These artificial genes are then constructed e.g. by total gene synthesis or by the use of synthetic genetic subunits. These genetic subunits correspond to structural sub-elements on the (poly)peptide level. On the DNA level, these genetic subunits are defined by cleavage sites at the start and the end of each of the sub-elements, which are unique in the vector system. All genes which are members of the collection of consensus sequences are constructed such that they contain a similar pattern of corresponding genetic sub-sequences. Most preferably, said (poly)peptides are or are derived from the HuCAL consensus genes: Vk1, Vk2, Vk3, Vk4, V11, V12, V13, VH1A, VH1B, VH2, VH3, VH4, VH5, VH6, Ck, Cl CH1 or any combination of said HuCAL consensus genes.

This collection of DNA molecules can then be used to create libraries of antibodies or antibody fragments, preferably Fv, disulphide-linked Fv, single-chain Fv (scFv), or Fab fragments, which may be used as sources of specificities against new target antigens. U.S. Pat. No. 6,300,064 discloses methods for making synthetic libraries.

In another embodiment, synthetic human antibodies have now been made by synthesis from defined V-gene elements. Winter (EP 0368 684 B 1) has provided a method for amplifying (by PCR), cloning, and expressing antibody variable region genes. Starting with these genes he was able to create libraries of functional antibody fragments by randomizing the CDR3 of the heavy and/or the light chain. This process is functionally equivalent to the natural process of VJ and VDJ recombination which occurs during the development of B-cells in the immune system. For example, repertoires of human germ line V_{H} gene segments can be rearranged in vitro by joining to synthetic “D-segments” of five random amino acid residues and a J-segment, to create a synthetic third complementarity determining region (CDR) of eight residues. U.S. Pat. No. 5,885,793 discloses methods of making such antibody libraries such as these.

Manipulation of the Oxidative State of the Periplasm

A preferred embodiment of practicing SECR™ involves assembly of the binding ensembles within the periplasm of a bacteria. Occasionally it helps to modify the oxidative state of the periplasm.

The secretory compartment of gram-negative bacteria, i.e., the periplasmic space, bears many similarities to the secretory compartments of eukaryotic cells, thus it is a promising milieu for the f unctional expression and cell-based selection of antibodies, particularly for secreted antigens, which form the majority of therapeutic targets. Since methods are available for the assembly and screening of much larger expression libraries in gram-negative bacteria such as E. coli, than in any other cell type, the E. coli periplasm should be uniquely suited for antibody selection. The only serious drawback of the bacterial periplasm is its oxidizing power, which is much stronger than that of the eukaryotic endoplasmic reticulum. As a result, when eukaryotic proteins are translocated into the periplasmic space, they often do not fold fast enough to avoid the formation of non-native disulfide bonds by the potent oxidase, DsbA, which arrests folding. Such disulfides can be reduced by reductases to allow folding to continue, but this often does not occur fast enough to save heterologous proteins from aggregation or proteolysis.

We have found that periplasmic expression of many eukaryotic proteins can be substantially improved by including thiol reagents such as reduced glutathione, dithiothreitol, N-acetyl cysteine, or β-mercaptoethanol in the growth medium (e.g., see U.S. patent application Ser. No. 09/764,163). These reagents diffuse readily into the periplasm and interact with the DsbA enzyme, thereby competitively reducing its activity toward protein thiols. For example, reduced glutathione may be included in the medium at concentrations ranging up to 5 mM before it becomes toxic. Within this range soluble periplasmic expression, as judged by western blotting, has been seen to increase steadily for a variety of heterologous proteins, including human and murine antibody single-chain Fv fragments, VH domains, VL domains, Fab fragments, other immunoglobulin-like domains such as the CD86 receptor, other receptors such as CD40, CD40 ligand, and the epidermal growth factor receptor (EGFR), and non-secreted proteins such as calmodulin. Thus, most eukaryotic proteins, particulary secreted proteins, can be functionally expressed, at least at moderate levels, in the E. coli periplasm by, where necessary, including a sulfhydryl reagent in the medium at an optimal concentration which may be empirically determined.

### Selection/Screening Systems

Fragment and subunit complementation systems can be used in the invention to select/screen for antibodies having desired properties (“complementation system”). In general, fragment complementation systems are comprised of a responder that is fragmented or separated into two (or more) parts that must reassociate to make a functional responder. The fragments/subunits of the responder are fused individually to members of a binding ensemble, and the reassembly of the responder is then driven by the direct or indirect interaction of the two binding ensemble members. In a preferred embodiment the binding ensemble is comprised of an antibody(s) and an antigen(s). Examples of fragment/subunit complementation systems that may be used in the invention are disclosed in U.S. Pat. Nos. 6,342,345, 6,270,964, 6,294,330, 5,503,977, 5,585,245, PCT patent application WO 00/71702, and Fields et al. Nature 340:245-247 (1989), Bai et al. Meth. Enzymol. 273:331-347 (1996), Luo et al. Biotechniques 22:350-352 (1997), which are hereby incorporated by reference in their entirety.

Reactivation-based molecular interaction systems (eg. RAIR™) can be used in the invention to select/screen for antibodies having desired properties (“reactivation system”). In general, reactivation-based molecular interaction systems are comprised of responders, inhibitors, reactivators, and binding ensembles of two or more members. The system has two complexes, one containing the responder, the inhibitor, and a binding ensemble member (the responder complex), and the other containing the reactivator and a binding ensemble member (the reactivator complex). The responder is inhibited in its complex, and docking of the reactivator complex to the responder complex by direct or indirect interaction of the binding ensemble members allows the reactivator to “reactivate” the responder by displacing the inhibitor. Typically, a responder complex comprises a responder molecule, an inhibitor of the responder, and a first binding ensemble member. The components of the responder complex may be arranged in various configurations by covalent or non-covalent linkages.In a preferred embodiment the binding ensemble is comprised of an antibody(s) and an antigen(s).

In a preferred reactivation system, molecular interactions can be detected by a process termed “reactivation of an auto-inhibited responder,” or “RAIR.” The RAIR systems comprise the following components: a responder complex and a reactivator complex. By auto-inhibited, we mean that the responder is directly linked to the responder so that the base state is automatically inhibited until the inhibitor is displaced and the responder activated by a reactivator complex. Where this linkage is by a covalent bond, the covalent linkages may further comprise a linker. A reactivator complex comprises a reactivator molecule to displace the inhibitor and a second binding ensemble member. Like the components of the responder complex, the reactivator and binding ensemble member may be linked either covalently or non-covalently.

Molecular interaction between the first and the second ensemble members can be detected by the following mechanism: the signal or activity of the responder in the responder complex is sequestered by the inhibitor present in the complex, i.e., the responder is auto-inhibited; when a reactivator complex is introduced, if the second ensemble member in the reactivator complex binds with sufficient affinity to the first ensemble member in the responder complex, the reactivator will be able to displace the inhibitor in the responder complex and lead to the so-called “reactivation of an auto-inhibited responder.” The detection of responder activity or signal indicates an interaction between the first and the second ensemble members.

Variations of the RAIR systems can be used for interaction mapping, improving the affinity of a first binding pair member, and isotropic selection of a plurality of binding molecules. In some variations, a third ensemble member may be used.

Examples of reactivation systems are disclosed in U.S. patent application Ser. No. 10/208,730 which is incorporated herein by reference.

Systems using molecular sensors activated by competition can also be used in the invention to select/screen for antibodies having desired properties. These systems are designated COMPACT™. In general, competitive activation systems are comprised of a binding ensemble, a responder, and an inhibitor. The responder is complexed with one binding ensemble member and the inhibitor is complexed to another binding ensemble member. The binding ensemble members, upon binding to one another, bring the responder and inhibitor together so that the responder is inhibited. Antibodies of the invention that disrupt the binding ensemble or inhibit binding ensemble formation and thereby activate the responder can then be selected. In a preferred embodiment, the binding ensemble is an antibody(s) and an antigen(s), and the “competitive activator” is an antibody. For example, the binding ensemble antibody might be a reference antibody, and the competitive activator may comprise a library of antibodies which compete with the reference for binding to the antigen. Examples of competitive activation systems that may be used in the invention are disclosed in U.S. patent application Ser. No. 10/076,845 which is incorporated herein by reference.

Such a system may further employ a “mask” to control the sensitivity of the system. These systems are described, e.g., in co-pending U.S. application Ser. No. 10/076,845, filed Feb. 14, 2002. A “mask”, in the context of a competitive activation system, refers to a molecule that has low affinity for a reporter or inhibitor, such that the mask does not bind appreciably at working concentrations unless it is tethered covalently to the reporter or inhibitor. The mask does not affect reporter activity only the binding of the inhibitor and vice versa. Control of the system with Masks permits a high-affinity inhibitor to be used without fear of increasing the background inhibition because its association rate constant is greatly reduced by the Mask without affecting the dissociation rate constant of the reporter-inhibitor complex, thereby reducing the overall affinity while retaining the stability of the high-affinity reporter-inhibitor complex.

### Applications

The interaction systems of the invention can be used to select or screen for antibodies that are fully human idiologs of a non-human reference antibody of interest, i.e., have equivalent binding specificity to that of the reference antibody. In preferred embodiments the human idiologs will have bioactivities and affinities comparable to or greater than those of the reference antibody.

Serial Epitope-guided Chain Replacement (SECR)

SECR (pronounced “seeker”) is a method for isolating human idiologs of non-human antibodies. Idiologs are antibodies which have the same binding specificity as a reference antibody, but which are not derived from the reference antibody. In SECR, the heavy chain and light chain variable regions (V-regions VH and VL, respectively) of the non-human reference antibody are replaced one at a time with human V-regions which have the same binding specificity as the reference antibody. In each step an original or previously-selected V-region is used as a “guide” for selection of a new companion V-region from a library of diverse human V-regions. Test antibodies comprising a non-human or previously selected human “guide” and new V-regions are selected or screened for their ability to compete with a “competitor” for antigen binding in one of the interaction assays of the invention, wherein a reporter is activated in proportion to the affinity and stability of the test antibody.

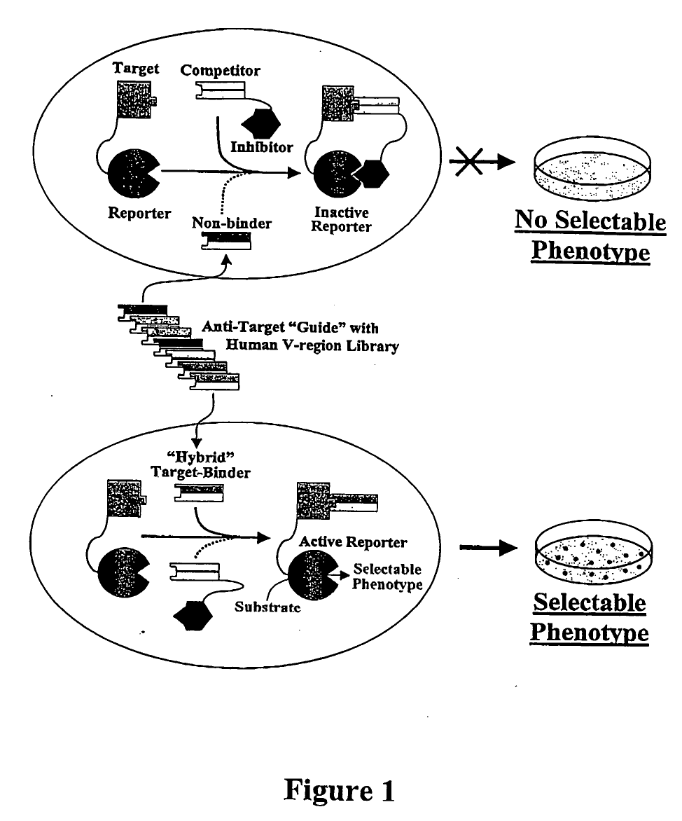

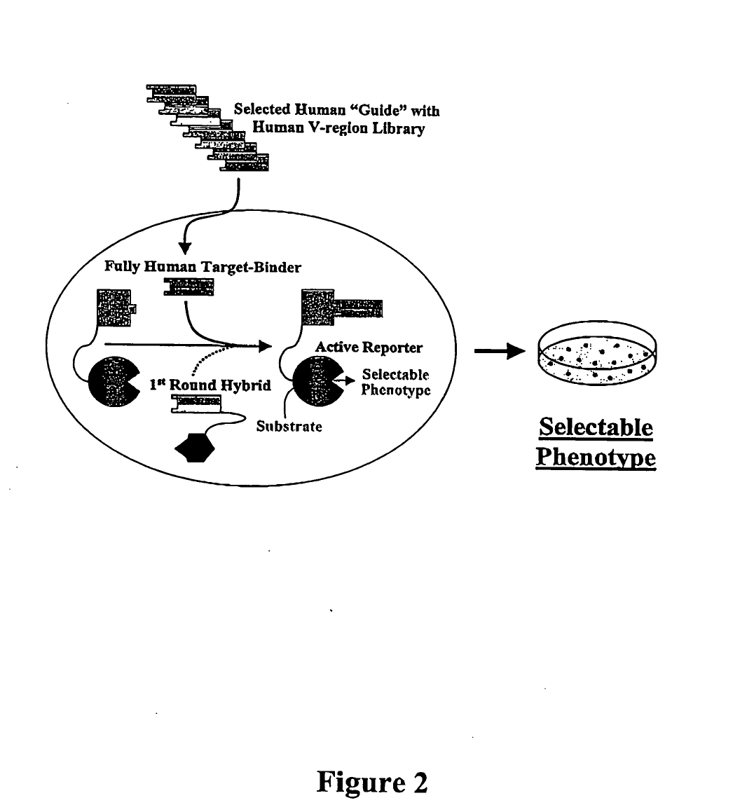

FIGS. 1 and 2 illustrate the 2-step process of guided selection of human idiologs of a reference antibody using a competitive reporter activation system such as those disclosed in U.S. patent application Ser. No. 10/076,845. In this illustration, the system components are expressed in cells. The reporter system is comprised of a protein which confers a selectable phenotype on the cells and a low-affinity inhibitor of the protein. Typically, the reporter is an enzyme which may confer a color phenotype or viability phenotype (i.e., the ability to grow under adverse conditions) on the cells. The target and a competitor, typically the reference antibody, are fused to the reporter and inhibitor, respectively, such that target-competitor binding causes the inhibition of the reporter, and failure of the cells to exhibit the selectable phenotype. These components are co-expressed in the presence of an antibody library in which each library member is comprised of the same V-region guide from the reference antibody and a member of a diverse population of human V-regions of the companion type (e.g., VH for VL guide). The V-regions of the reference antibody may be replaced in either order. Test antibody library members that bind to the target will compete with the competitor to produce the selectable phenotype in proportion to their affinities and stabilities.

In a preferred embodiment the reference antibody is used as the competitor in SECR. However, any molecule that has the desired binding specificity, i.e., binds to the desired epitope on the target antigen of interest, may be used as the competitor in SECR. For example, derivatives of the reference antibody such as single-chain Fv (scFv) or Fab fragments may be used as the competitor, or natural ligands of the target antigen which bind to the desired epitope may be used as the competitor. Alternatively, an artificial binding protein such as a peptide selected from a random peptide library which may or may not be displayed on the surface of a scaffold protein such as thioredoxin may be used as the competitor. Of course, antibodies selected in a previous round of V-region replacement may be used as the competitors for subsequent rounds of V-region replacement.

The primary function of the reference antibody is to provide the guide chains whereby the selection of new V-regions is constrained to the same epitope in the same orientation as that bound by the reference antibody. In this capacity the reference antibody ensures that selected V-regions will retain as much of its binding specificity as possible. Thus, the antibodies obtained from the first round of selection will all have the same V-region from the reference antibody and another V-region from a library of diverse human V-regions of the companion chain type (i.e., heavy or light), and they will bind to the same epitope as the reference antibody, preferably in the same orientation. In the second round, the reference antibody V-region present the hybrid antibodies selected in the first round is replaced from a library of diverse human V-regions of the same chain type. The antibodies obtained from this round of selection are fully human idiologs, binding to the same epitope on the same antigen as the reference antibody. Additional rounds of replacement may be performed in order to obtain the best pair of V-regions from the libraries employed with respect to affinity and stability.

The libraries used for serial chain replacement can be any library disclosed above or that is well known in the art, including for example, human repertoire antibody libraries, or human germline antibody libraries with synthetically randomized CDRs, or human germline antibody libraries recombined with natural CDR repertoires. Libraries of other species may be used where antibodies are desired for therapeutic use in that species, e.g., bovine, porcine, ovine, feline, canine, or other mammal libraries.

The successful application of hybridoma technology for the last 27 years has produced hundreds of murine antibodies with unique specificities and bioactivities, which could be of great therapeutic value. In addition, thousands of new targets for antibody-based therapy are expected to emerge from the human genome, and most of these will be validated with non-human antibodies generated with hybridoma technology. However, many therapeutic applications require repeated administration, and the efficacy of non-human antibodies tends to decline under such regimes due to their inherent immunogenicity. At the same time the risk of anaphylaxis increases, especially when large doses are needed. In many cases it has not proved possible to replace these antibodies with human ones using the in vitro display methods or mice genetically engineered to produce human antibodies, perhaps due to the epitope biases inherent in such methods and systems. The only alternatives have been various related humanization strategies, the most successful of which involve grafting the CDRs of the non-human antibody onto human germline frameworks selected for their structural homology to the non-human framework (Winter, supra). Typically this process results in a substantial losses in affinity, at least some of which can be regained by restoring murine residues at key positions, and making other mutational adjustments in the structure based on sophisticated modeling techniques combined with trial and error. In many cases, however, the full affinity of the original non-human antibody has not been recovered, and the product is not fully human. Thus, it may retain some immunogenicity.

With the present invention, the target epitope and one V-region of the non-human “reference” antibody are used to guide the selection of new human V-regions from large libraries, with the highest affinities present in those libraries, and with the same orientation on the target epitope for maximum retention of bioactivity. This is accomplished by employing a competitive activation system, such as those disclosed in U.S. patent application Ser. No. 10/076,845, in which the target antigen and the reference antibody, are genetically linked to reporter and inhibitor, respectively, such that binding of the reference antibody to the target antigen causes the inactivation of the reporter. These components are co-expressed in cells, preferably in the secretory compartments, along with a library of “test” antibodies comprised of the guiding V-region from the reference antibody and a library of human V-regions of the opposite type. The reference antibody thus serves as a “competitor”, against which the test antibodies compete for binding to the target antigen. Test antibodies which compete successfully, prevent docking of the inhibitor to the reporter, thereby activating the reporter and producing the selectable phenotype in proportion to their affinities for the antigen.

Only in cell-based competitive systems is the selectability of target-binding antibodies proportional to affinity at all times, allowing rapid identification of selected antibodies with the highest affinities for the target antigen. In non-competitive systems, such as the display technologies, or cell-based complementation systems where the selectable phenotype is produced directly by antigen—test antibody interaction, affinity is only limiting for selection when antibody and antigen concentrations are in the range of equilibrium dissociation constants (K_{d}) corresponding to desired affinities. However, in most systems such concentrations are too low to allow robust generation of selectable signals or phenotypes. For example, for therapeutic antibodies, affinities corresponding to K_{d} less than ˜1 nM are desired. However, for most detection systems such concentrations are generally too low to confer readily detectable phenotypes on cells, or to generate readily detectable signals in vitro. As a result, under conditions of robust signal or phenotype generation in non-competitive systems, affinity is generally not limiting, and affinities corresponding to K_{d} less than ˜0.1 μM cannot be reliably distinguished. Only in cell-based systems where each library member is forced to compete one-on-one with a common competitor is the ability of any test antibody to cause the activation of the reporter always proportional to its affinity for the antigen regardless of its concentration. Thus, at concentrations required for robust signal or phenotype generation, in the cell-based competition system, the strength of the signal/phenotype is still proportional to the affinity of the test antibodies, allowing the highest affinity antibodies to be readily identified.

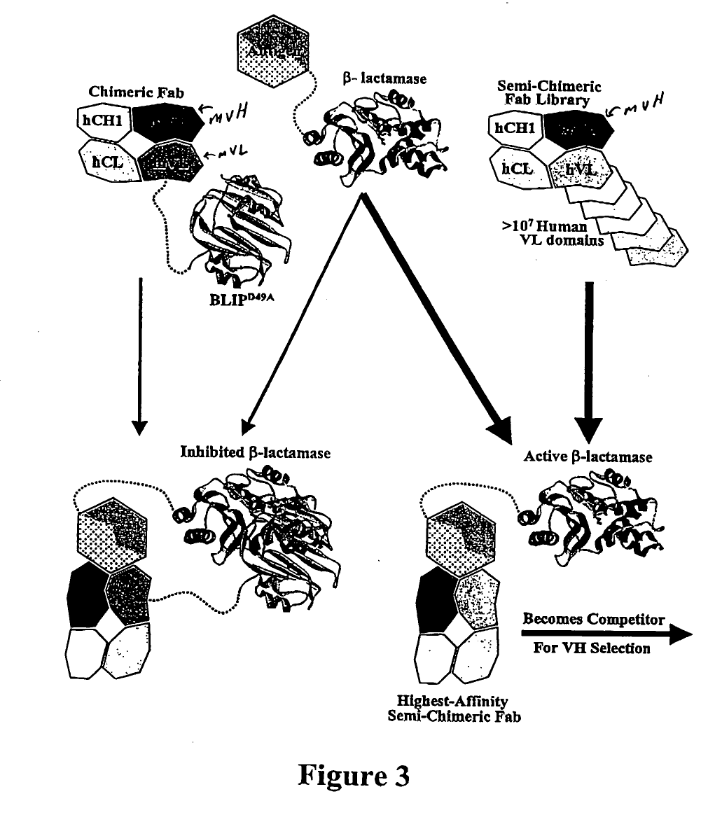

In the preferred embodiment of the β-lactamase competitive activation system a fragment of the reference antibody, such as a single-chain Fv or Fab, serves as the competitor, and is therefore expressed as a fusion to the carboxyl terminus of the β-lactamase inhibitor, BLIP, in the periplasmic space of E. coli cells. In this preferred orientation BLIP also serves as a translocation chaperone, thereby often improving the expression of the reference antibody. The BLIP-competitor fusion is co-expressed with the target antigen fused to either terminus of β-lactamase, so that the antigen-competitor interaction brings BLIP and β-lactamase together, causing the inactivation of the latter and failure of the cells to grow on β-lactam antibiotics such as ampicillin (see FIG. 3). The affinity of the BLIP-β-lactamase interaction is reduced by mutations such as BLIP-D49A or β-lactamase E104K, N, Q, or A, so that the antigen-antibody interactions are required for β-lactamase inhibition. The desired orientation of the antigen/β-lactamase fusion is determined by which produces the most efficient β-lactamase inhibition upon antigen-competitor binding.

When a test antibody library is co-expressed in the same cells in Fab or scFv format, test antibodies which compete with the competitor for binding to the antigen will activate β-lactamase in proportion to their affinities. For any selection of Fabs with a competition system in which both competitor and test antibodies are expressed as Fabs, only one chain can be varied at a time to avoid the mixing of competitor chains and test chains. Thus, for light chain (LC) selection, the LC of the competitor Fab must be linked to BLIP, and the competitor and test Fabs will share the heavy (Fd) chain of the parent Fab. If the Fd chain were fused to BLIP for LC selection, or if both chains were varied, then selectable chains would also increase inhibition and would therefore fail to produce a net activation of β-lactamase.

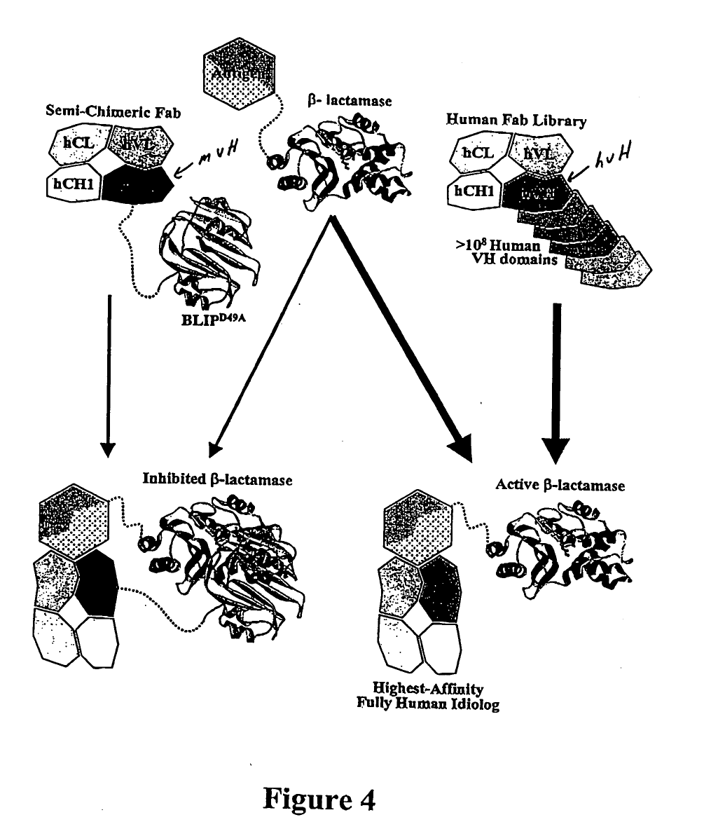

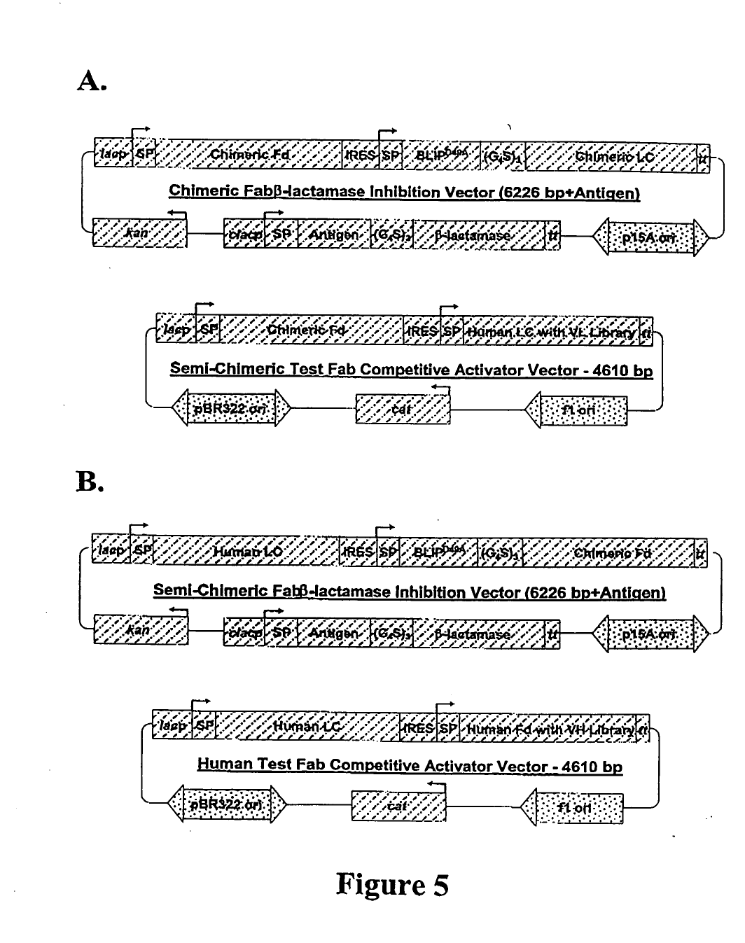

Typically, non-human Fabs can be chimerized with little loss in affinity by fusing the non-human V-regions to homologous human constant regions. In one embodiment the chimeric Fab is then expressed with the β-lactamase inhibitor, BLIP, fused via flexible linker to the amino terminus of either VH or VL, depending on which V-region is to be replaced first. As before, the antigen is fused to either terminus of β-lactamase, so that binding of the chimeric Fab to the antigen docks BLIP to β-lactamase, inactivating the latter. New Fabs are then selected from test Fab libraries for their ability to compete with the chimeric reference Fab for antigen binding, and thereby activate β-lactamase. This strategy for conversion of a mouse/human chimeric Fab to high-affinity fully human idiologs is illustrated in FIGS. 3 and 4, and the vectors for expression of the components of the system are shown in FIG. 5.

The critical step in the process is VL-guided VH selection because the VH is expected to contribute most of the affinity of the antibody, and also because VL is usually the weaker guide due to its generally lower affinity than that of the VH. Thus, it could be argued that whichever VL, i.e., non-human reference VL or selected human VL, has the higher affinity would make the best guide for human VH selection. By this rationale, VH-guided VL selection should be done first, because if a human VL is obtained that confers higher affinity on the hybrid antibody than that of the reference antibody then the selected human VL should be used to guide human VH selection. On the other hand, if the best selected hybrid has a lower affinity for the antigen than the non-human reference antibody, then the VL from the latter should be used to guide VH selection. Either way, the best selected high-affinity human VH idiolog can then provide a strong orientation guide for selection of a high-affinity human idiolog for the VL domain. In this way, by preserving not just the epitope, but the orientation of the antibody on the epitope as well, the full biological activity of the original antibody should be faithfully preserved. For purposes of illustration, the process will be described for reference VH-guided human VL selection as the first step in the selection of human idiologs with both test antibodies and competitors in Fab format.

It is usually necessary to fuse BLIP to the amino-termini of the V-regions in the Fab format to ensure steric accessibility of β-lactamase and BLIP in the presence of the bulky Fab fragment (57 kDa and elongated). This has the added benefit of using a naturally secreted E. coli protein to ensure efficient translocation of the Fd or LC to the bacterial periplasm. When the VL domain is to be replaced first with test antibodies also in Fab format, the competitor must be made by fusing BLIP to the mouse VL domain in the chimeric reference Fab for the selection to work. The test Fab library will be semi-chimeric, having only one of four domains from the mouse, and differing from the reference Fab in only one of four domains, that being represented by a diversity of V-regions. Both the test Fab library and the chimeric Fab competitor will share the same Fd fragment, though it may be expressed from two genes to ensure that it is not limiting. The Fabs may be expressed from dicistronic transcripts. However, to ensure that the common chain shared by competitor and test Fab is not limiting it may be expressed from the upstream cistron in both cassettes (see FIG. 5). The V-region domains may be derived from non-immune human repertoires, or they may have synthetic random sequences in one or more of their CDRs, or they may combine both sources of diversity, or they may be recombined from natural framework and CDR repertoires. In any case, it is preferable to have at least 10⁸ different VH domains or 10⁷ different human VL domains in the test Fabs.

The β-lactamase activity in each cell of the system will be proportional to the affinity of the semi-chimeric test Fab expressed in that cell. To enrich the selection for semi-chimeric Fabs which have affinities at least comparable to or higher than the parent chimeric Fab, the initial selections should be performed by plating the cells on the maximum permissive antibiotic concentration for cells expressing the chimeric reference Fab as both competitor and test Fab. This is determined experimentally as the highest antibiotic concentration on which the plating efficiency (PE, percent of cells plated forming colonies) of cells expressing the reference Fab as both competitor and test Fab is at least 10% but preferably 100%.

The sensitivity of the system is proportional to the increase in PE per unit increase in affinity. This may be optimized by using inducible promoters to adjust the expression levels of the components of the system to achieve the maximum difference in PE between cells expressing the reference Fab as the test Fab and cells expressing the test Fab library (see FIG. 5). Typically, this difference should be at least 1,000-fold on the highest antibiotic concentration on which the reference Fab plates quantitatively. Examples of inducible promoters include the lactose operon promoter, which is activated by isopropyl thiogalactoside (IPTG), and the arabinose operon promoter, which is activated by arabinose (Sambrook and Russell, eds, Molecular Cloning: A Laboratory Manual, 3rd Ed, vols. 1-3, Cold Spring Harbor Laboratory Press, 2001). Ideally, the antigen—β-lactamase fusion should be expressed at levels which allow robust antibiotic resistance when uninhibited, but which are below the reference Fab-antigen K_{d} to allow efficient competitor-test Fab competition, and which can be inhibited efficiently by competitor-BLIP levels which are below the threshold for detectable background inhibition, i.e., below the K_{d} of the β-lactamase—BLIP interaction. The test Fabs should be expressed at levels which give the maximum competitive activation by the reference Fab when expressed as the test Fab.

With a semi-chimeric test Fab library containing a human VL diversity of at least 10⁷, at least 10⁸ double transformants should be plated on the selecting antibiotic. Depending on the PE of cells expressing the reference Fab as both test Fab and competitor, the test Fab constructs from the resulting colonies should be rescreened to eliminate library background colonies. For example, with a PE ratio of 1000 (reference Fab over library), i.e., library background PE of ˜0.1%, stable positives having PEs of ˜10-100% should be enriched ˜100-1000-fold over background with each rescreening, and stochastic positives should be eliminated after two rescreenings.

Since in the first round of selection each semi-chimeric Fab contains an antigen-specific VH to guide the selection of human VL domains, false positive selection is disfavored because direct inhibition of reporter-inhibitor interaction by the Fab would be hindered by the VH. The selected semi-chimeric Fabs can be rank-ordered for affinity by rescreening on increasing concentrations of antibiotic. Those clones exhibiting the highest levels of antibiotic resistance will generally be expressing the highest affinity Fabs. It is possible for lower-affinity Fabs to be selected by virtue of unusual VL stability which allows them to accumulate to higher intra-cellular concentrations. However, this is disfavored by using very low expression levels in the system, under which condition most Fabs are sufficiently stable that additional stability confers little selective advantage. Also, Fab stability is strongly influenced by the light chain and the heavy chain constant region, which are the same for all library members. Selected clones can also be rank-ordered for affinity by antibody-capture ELISA using culture supernatants which have been diluted to normalize antibody concentrations after the latter have been determined by immunochemical techniques (for methods see Harlow and Lane, Antibodies A Laboratory Manual Cold Spring Harbor Laboratory Press, Cold Spring Harbor, 1988). The affinities and kinetic constants of the highest-affinity semi-chimeric Fabs can be determined by surface plasmon resonance (Faigerstam et al. (1992) J Chromatog 597: 397-410).

Once the highest affinity semi-chimeric Fabs have been identified, the selected human light chains become the guides for selection of high-affinity human VH domains to obtain fully human idiologs (see FIG. 4). The semi-chimeric Fab may be used as the competitor for this selection by fusing BLIP to the amino terminus of the chimeric Fd fragment. The human LC guide is shared by the competitor and the fully human test Fab library. To ensure that the shared LC is not limiting for human VH selection, it is expressed from the upstream cistron in both of the dicistronic Fab expression cassettes. As before, the VH library may be derived from non-immune human repertoires, or it may have synthetic random sequences in one or more of their CDRs, or it may combine both sources of diversity. In either case, it is preferable to have at least 10⁸ different human VH domains in the test Fabs. The selections are performed essentially as described for VL selection, except that with a human VH library of at least 10⁸ different molecules, at least 10⁹ doubly transformed cells should be screened. Again, the highest affinity fully human Fabs can be determined by plating on increasing concentrations of antibiotic, and the affinities of these Fabs can be determined by surface plasmon resonance.

In an alternative embodiment of the invention, surrogate molecules which bind the same epitope on the target antigen as that bound by the reference antibody may be used as the competitor in place of the reference antibody. Surrogate competitors may be derived from the reference antibody, or they may be completely different. For example, a natural ligand of the target antigen may be used as the competitor if it binds the same epitope as the reference antibody. Use of a surrogate competitor may be warranted for example, when the reference antibody does not express well or function well when fused to components of the reporter system. Also, use of a surrogate competitor avoids complications which may arise when two Fabs are expressed in the same cell. For example, when the test chain and competitor chain-inhibitor fusion compete for the common chain (which is shared by both the test Fab and the competitor Fab), false positive reporter activation may occur if the test chain out-competes the competitor chain for binding to limiting amounts of the common chain. In this case, less competitor Fab would form, thereby docking less inhibitor to the reporter, leading to reporter activation without antigen binding by the test Fab. This source of false positives can be avoided by using a single-chain Fv (scFv) fragment of the reference Fab as an intermediate in the selection because the scFv carries its own copy of the common chain V-domain, and thus, would not need to compete with the test or competitor chain for the common chain.

In this embodiment, an scFv fragment of the reference Fab is used in place of the reference Fab as the competitor in the first round of selection, so that only the test molecules are in the Fab format. However, often scFv versions of antibodies have reduced affinities and/or do not express well, and therefore might not perform adequately as competitors. In such cases, the scFv format could be used for selection of the first human V-region domain, and the Fab format retained for the competitor. For example, the reference antibody VH could be paired with a human VL library in scFv format, and expressed from the “competitive activator” vector shown in FIG. 5A in cells expressing the chimeric Fab β-lactamase inhibition vector shown in FIG. 5A. The non-human reference VH would serve as a strong guide for selection of high-affinity human VL domains. The resulting scFv could then be expressed as the BLIP fusion to provide a high-affinity competitor for human Fd selection in Fab format.

Alternatively, both steps of the selection process could be performed with the test molecules in scFv format. The highest-affinity VL domains selected in the first round would serve as guides for the selection of high-affinity VH domains in the second round. The resulting scFvs could then be converted, if desired, to the human Fab format by the addition of human C-region domains, or to full-length immunoglobulins (Ig) by the addition of C-regions and Fc domains.

In a preferred embodiment of SECR using scFv fragments, the V-domains are typically linked in the order VH-VL by a flexible linker, e.g., (Gly₄Ser)₃₋₅, and the hybrid (e.g., mouse/human) scFv test library is expressed from the competitive activator vector shown in FIG. 5A, in place of the semi-chimeric test Fabs. The preferred stringency of selection would be the same as that used for Fab selection, i.e., the level of activation produced by the reference Fab when expressed as the test Fab. Under these conditions, and when scFv expression is comparable to test Fab expression, then the selection of scFv with affinities at least comparable to that of the reference Fab is favored. As before, selection is guided to the original epitope by both the reference V-domain, and by the requirement for competitive inhibition of reference Fab binding for selectable activation of the reporter. Since, as before, the reporter activity will be proportional to the affinity of the test scFv, the selected epitope-binding hybrid scFv can be rank-ordered with respect to affinity by the strength of the phenotype conferred on the cells by the reporter, e.g., antibiotic resistance when the reporter is β-lactamase.

The highest-affinity epitope-binding hybrid scFv are then used as competitors (i.e., after being modified into BLIP fusions) for selection of the second chain, preferably in Fab format. In the preferred embodiment, the hybrid scFv are sub-cloned into the β-lactamase inhibition vector shown in FIG. 5B, in place of the semi-chimeric Fab, for expression as the BLIP fusion. Each hybrid scFv is then tested for efficient inhibition of β-lactamase by scFv-antigen interaction, and any false positive scFv, e.g., BLIP- or β-lactamase-binders, are eliminated. For second chain selection in Fab format, the human V-domain from each epitope-binding hybrid scFv is fused to the appropriate human constant region domain and co-expressed with a library of the companion chain from the Human Test Fab Competitive Activator Vector (see FIG. 5B), as in the original all-Fab system illustrated in FIG. 4. The preferred stringency of selection would exclude anything less than the level of activation produced by the original chimeric reference Fab when expressed as the test molecule, competing against the hybrid scFv as the BLIP fusion. Under these conditions, the selection of fully human test Fabs with at least comparable affinities for the original epitope to that of the original chimeric Fab is favored. Since, as before, the reporter activity will be proportional to the affinity of the test Fab, the selected human Fabs can be rank-ordered with respect to affinity by the strength of the phenotype conferred on the cells by the reporter. The Fabs can then be purified, and affinities determined by surface plasmon resonance.

Membrane-bound antigens, including human receptors with multiple transmembrane segments, can also be accommodated as target antigens in SECR. Membrane proteins may be fused to the reporter system via flexible linkers at either terminus, so long as the reporter is on the periplasmic side of the membrane. However, fusion of native bacterial reporters such as β-lactamase to the amino terminus has the added benefit of providing an efficient translocation chaperone for insertion of the target antigen into the bacterial membrane. Additional membrane-spanning segments (e.g., Gurezka et al., 1999, J Biol Chem 274:9265-9270) may need to be added to the amino and/or carboxyl terminus of proteins whose termini are normally on the cytoplasmic side of the membrane to facilitate insertion into the bacterial plasma membrane in the proper orientation, and to ensure placement of the reporter on the periplasmic side. Additional membrane spanning segments which reverse the orientation of the downstream sequence may be derived by duplication of the terminal membrane-spanning segments in the protein. As in the preferred embodiments for SECR described above, the competitor, which may be either the reference antibody, or a surrogate thereof, is fused via flexible linker to either terminus of the inhibitor, and selections are performed in the E. coli periplasm as described above.

All publications and patent applications cited in this specification are herein incorporated by reference as if each individual publication or patent application were specifically and individually indicated to be incorporated by reference.

### EXAMPLES

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### Example 1

### Conversion of a Mouse Anti-CD40 Antibody to a Higher Affinity Human Idiolog

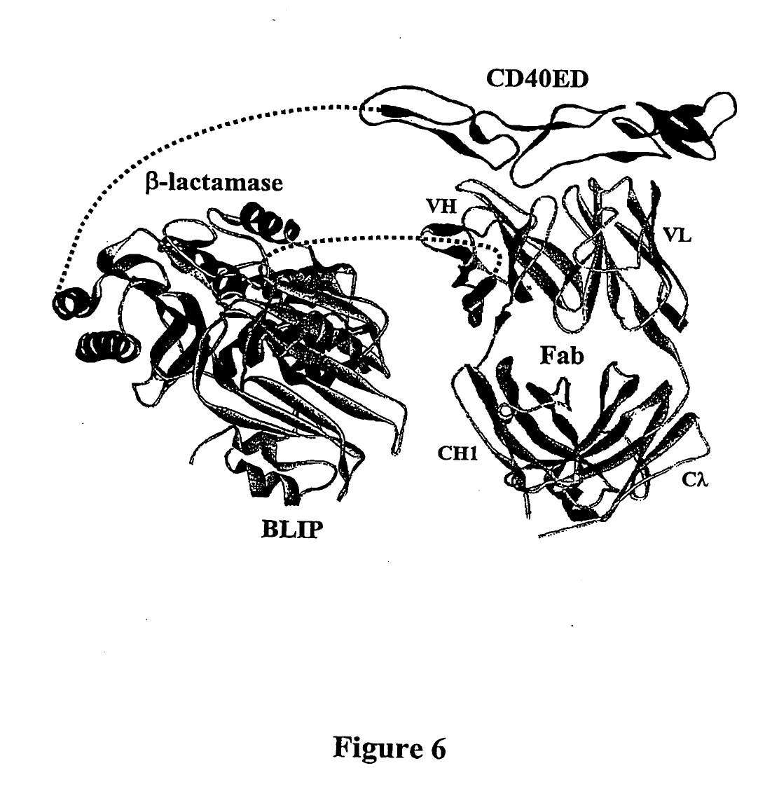

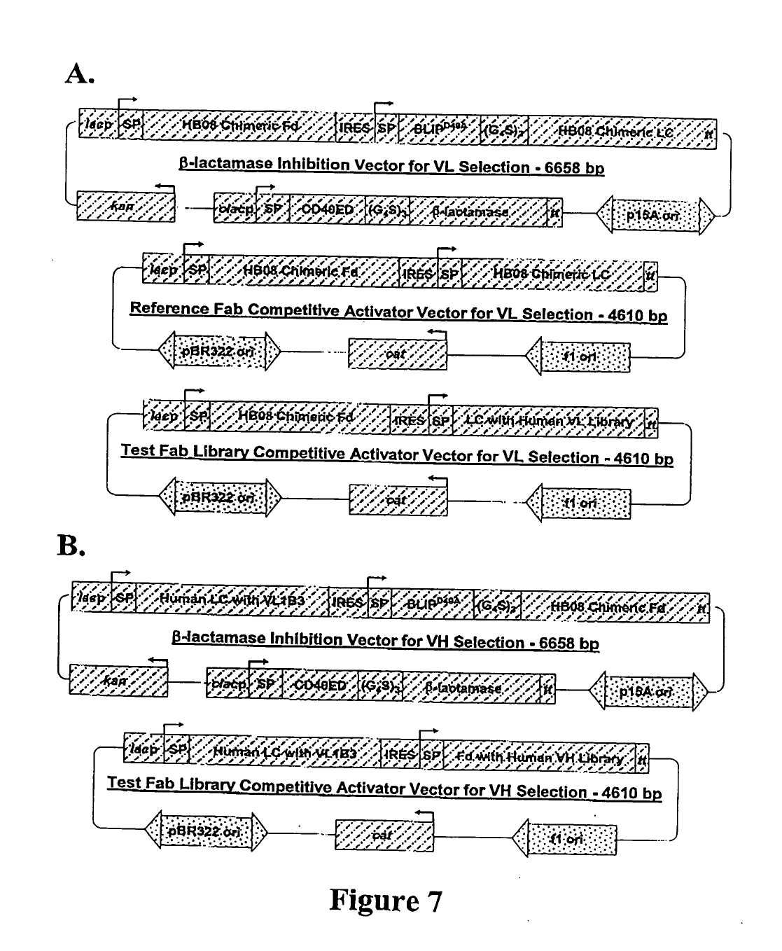

This example demonstrates the utility of the invention for conversion of non-human antibodies to human idiologs which bind precisely the same epitope, and with comparable or higher affinity. The V-regions of a murine monoclonal antibody, designated HB08, that binds to human CD40 antigen, were replaced by human V-regions to create a fully human Fab which bound the same epitope with comparable affinity. The docking of BLIP to β-lactamase by the binding of a Fab to the 13.5 kDa extra-cellular domain of human CD40 is illustrated in FIG. 6. The variable region coding sequences of HB08 were recovered from a hybridoma cell line by RT-PCR using commercial primers for the consensus sequences of murine VH and VL framework 1 framework 4 regions, respectively (Pharmacia Corp.; Larrick and Balint, 1994, in Antibody Techniques, Malik and Lillehoj, Eds., Academic Press, New York, pp. 103-114). After sequencing, the mature V-region coding sequences were subcloned into the β-lactamase inhibition and competitive activator vectors shown in FIG. 7 for expression as Fab fragments with human CH1 and CL constant regions.

The Fabs in the two vectors were expressed from dicistronic transcripts. Since the VL domain was to be replaced first, the common Fd chain was expressed from the upstream cistrons to ensure that it was in excess over the competing light chains. The coding sequence of the human CD40 extra-cellular domain (CD40ED; Bajorath and Aruffo, 1997, Proteins: Struct, Funct, Genet 27:59-70) was recovered and amplified by RT-PCR from a commercial preparation of mRNA from peripheral blood lymphocytes, and inserted into the β-lactamase inhibition vector for expression of CD40 fused to the amino terminus of β-lactamase (see FIG. 7A). The HB08 chimeric Fab was expressed from the same vector with BLIP fused to the amino terminus of the light chain. The foregoing and following manipulations of recombinant DNA cloning and expression in bacteria were carried out using methods well known in the art and described in detail in, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997.

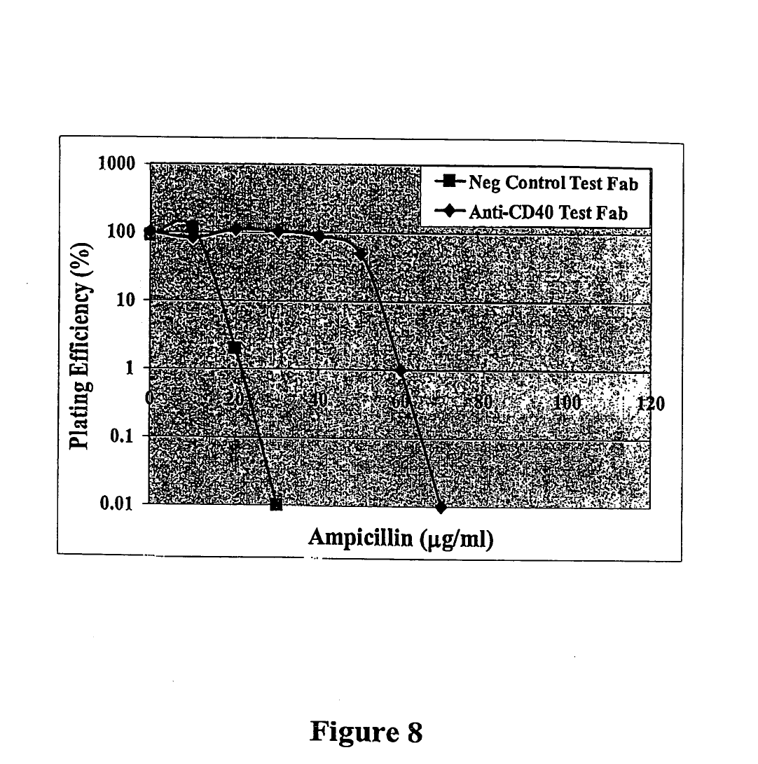

To establish optimum selection conditions we looked at how well the HB08 Fab competed with itself for competitive activation of β-lactamase. To accomplish this, the β-lactamase inhibition vector and the reference Fab competitive activator vector shown in FIG. 7A were co-introduced into E. coli TOP10F′ cells by high-voltage electroporation and plated onto solid medium (2×YT) containing increasing concentrations of ampicillin and 10 μM IPTG, the transcription inducer for the BLIP-competitor fusion and the test Fabs. After overnight growth at 33° C., plating efficiencies were determined and compared with those of cells expressing a negative control Fab as the test Fab. The results are shown in FIG. 8. As ampicillin increased, the plating efficiencies of cells expressing the negative control dropped off rapidly to ˜0.01% on 30 μg/ml, whereas cells expressing the HB08 chimeric Fab continued to plate at nearly 100% efficiency out to 50 μg/ml ampicillin, where the difference in plating efficiency was nearly 10,000-fold. This indicated that at 50 μg/ml ampicillin Fabs that bound CD40 with affinities comparable to HB08 would be enriched ˜10,000-fold relative to non-binding Fabs.

The coding sequence of the original murine VL domain in the light chain on the competitive activator vector was then replaced with a human non-immune repertoire of ˜10⁷ VL domains, obtained by RT-PCR from the peripheral blood of voluntary donors (Stanford University Blood Center). About 10⁸ transformants, each expressing the BLIP—reference Fab fusion, the CD40—β-lactamase fusion, and one semi-chimeric test Fab containing a VL from the human repertoire, were plated onto 50 μg/ml ampicillin. About 10⁴ colonies resulted, consistent with a background plating efficiency of ˜0.01%. The colonies were removed from the plates, and the semi-chimeric Fab-encoding activator plasmids were isolated and retransformed into fresh cells containing the β-lactamase inhibition plasmid. About 10⁵ transformants were replated onto solid medium containing 50 μg/ml ampicillin and 10 μM IPTG, and ˜160 colonies appeared after overnight growth. All 160 colonies were then screened by antibody capture ELISA for binding of the semi-chimeric Fabs they expressed to immobilized CD40 (for methods see Harlow and Lane, Antibodies A Laboratory Manual Cold Spring Harbor Laboratory Press, Cold Spring Harbor, N.Y. 1988).

The recombinant CD40 extra-cellular domain contained a carboxyl terminal His₆ tag, by which it was purified using immobilized metal ion affinity chromatography (IMAC; Janknecht et al., 1991, Proc Natl Acad Sci USA 88:8972-6). Purified CD40 was immobilized in the wells of 96-well polystyrene microtiter plates at, typically 100-500 ng per well. Each clone was grown in suspension culture overnight with selection for the activator vector only (chloramphenicol) and 1 mM IPTG, and vigorous shaking to release the Fab into the medium. The culture supernatants were then assayed for CD40-binding activity by ELISA. The selected Fabs contained a carboxyl terminal epitope tag by which they could be detected using an anti-tag antibody conjugated to horse radish peroxidase (HRP). CD40-bound Fabs were detected using a chromogenic HRP substrate.

Of the 160 selected clones, 13 were positive in ELISA. Many of the negative clones produced little detectable Fab by immuno-blot analysis. This is explained by the fact that while most Fabs express well under the low-expression conditions of the selection process, many cannot tolerate the higher expression levels required to produce enough material for ELISA. Upon sequencing, 6 of the 13 positive clones had the wild-type HB08 VH domain. This occurred because the HB08 VH was originally present in the vector into which the human VH library was inserted, and the vector preparation for library insertion must have been contaminated with uncut or single-cut vector. The remaining seven clones had only three unique sequences, two of which appeared three times each, and one of which appeared once. Table I summarizes the characteristics of the semi-chimeric Fabs containing each of these three selected human VL domains with respect to expression, antigen-specific antibiotic resistance, and performance in antibody capture ELISA after purification by immuno-affinity chromatography.

TABLE I
Characteristics of Semi-chimeric Anti-CD40 Fabs Containing
Human VL Domains Selected by Epitope-guided Chain
Replacement.

| Human VL | | | | | |
|---|---|---|---|---|---|
| in the Semi- | | | | | |
| Chimeric | | Ampicillin Resistance^{b.} | | | |
| Fabs | Expression^{a.} | 25 μg/ml | 50 μg/ml | 100 μg/ml | ELISA^{c.} |

| | | | | | |
|---|---|---|---|---|---|
| HB08 | +++ | 100% | 80% | <0.01% | 1.62 |
| Neg. | +++ | 0.2% | <0.01% | <0.01% | 0.076 |
| Control | | | | | |
| Fab | | | | | |
| VL1B3 | ++ | 85% | 100% | 0.1% | 1.34 |
| VL2B2 | +++ | 100% | 50% | <0.01% | 1.88 |
| VL2F12 | ++ | 75% | 15% | <0.01% | 0.93 |

^{a.}Expression of each selected human VL in the context of the semi-chimeric Fab was assessed by immunoblotting PAGE-separated proteins in culture supernatants of cells expressing the Fabs. Fd chains contained C-terminal Flag tag, and were detected with anti-Flag antibody.
+ denotes an arbitrary unit of band intensity on the developed blot.
^{b.}Ampicillin resistance conferred by each selected human VH was determined by co-expressing each semi-chimeric Fab with the BLIP-HB08 fusion and the CD40 - β-lactamase fusion in E. coli cells, and scoring the percentage of cells forming colonies on the indicated concentrations of ampicillin after overnight growth at 33° C.
^{c.}Antibody Capture ELISA was performed with 1 μg of purified CD40 immobilized in each well, and 0.1 μg of purified Fab per well. Values represent the optical absorbance at 490 nm after labeling captured Fab with anti-Flag antibody conjugated to alkaline phosphatase and developing with chromogenic substrate.

All three selected human VL domains were highly homologous to the murine HB08 VL, including >50% homology in CDR3. When adjusted for expression levels VL1B3 appeared to have the highest affinity by both ELISA and ampicillin resistance, and VL1B3 compared favorably with the HB08 Fab. However, VL2B2 expressed much better than VL1B3 and also had an apparent affinity that compared favorably with HB08. Thus, VL2B2 may be more robust for in vitro applications. Nevertheless, the high-affinity VL1B3 light chain was used in Fab format to select a companion human VH domain from a human VH library fused to a human Cγ1 constant region domain. The VH library in Fab format was made by essentially the same procedure, and from the same source material as was used for the VL library.

For VL1B3-guided VH selection, BLIP was fused to the amino terminus of the HB08 Fd chain in the competitor Fab, which now contained the VL1B3 light chain. The selection was carried out by essentially the same procedure as was used for VH selection with comparable results. Four human VH domains were obtained which coordinated with VL1B3 in Fab format to activate β-lactamase by competing with. Table II summarizes the characteristics of the fully human Fabs containing each of these four selected human VH domains with respect to expression, antigen-specific antibiotic resistance, and performance in antibody capture ELISA after purification by immuno-affinity chromatography. At least one of the selected VH domains, designated VH1D5, supported an affinity of the fully human Fab which was at least comparable to that of the HB08 chimeric Fab, though it did not express as well. VH3B4 expressed at least as well as HB08, but had slightly lower affinity. Interestingly, the sequences of the four VH domains showed little significant homology to the HB08 VH. However, they showed considerable homology to one another, except in CDR3, all being derived from the same germline VH gene.

TABLE II
Characteristics of Fully Human Idiologs of the HB08
Anti-CD40 Chimeric Fab.

| Selected | | | | | |
|---|---|---|---|---|---|
| Human | | | | | |
| VH in | | Ampicillin Resistance^{b.} | | | |
| the Test Fab | Expression^{a.} | 25 μg/ml | 50 μg/ml | 100 μg/ml | ELISA^{c.} |
| HB08 | +++ | 100% | 100% | <0.01% | 1.39 |
| Neg. | +++ | 4% | <0.01% | <0.01% | 0.051 |
| Control Fab | | | | | |
| VH1D5 | ++ | 85% | 100% | 0.05% | 1.24 |
| VH2E12 | +++ | 100% | 21% | <0.01% | 0.88 |
| VH2H8 | ++ | 75% | 9% | <0.01% | 0.63 |
| VH3B4 | +++ | 100% | 46% | <0.01% | 0.95 |

^{a.}Expression of each selected human VH in the context of the fully-human Fab was assessed by immunoblotting PAGE-separated proteins in culture supernatants of cells expressing the Fabs. Fd chains contained C-terminal Flag tag, and were detected with anti-Flag antibody.
+ denotes an arbitrary unit of band intensity on the developed blot.
^{b.}Ampicillin resistance conferred by each selected human VH was determined by co-expressing each human Fab with the BLIP-HB08Fd/VL1B3LC fusion and the CD40 - β-lactamase fusion in E. coli cells, and scoring the percentage of cells forming colonies on the indicated concentrations of ampicillin after overnight growth at 33° C.
^{c.}Antibody Capture ELISA was performed with 1 μg of purified CD40 immobilized in each well, and 0.1 μg of purified Fab per well. Values represent the optical absorbance at 490 nm after labeling captured Fab with anti-Flag antibody conjugated to alkaline phosphatase and developing with chromogenic substrate.

The present result does not constitute humanization of a non-human antibody as it is understood in the art, i.e., the process of mutating a non-human antibody to make it appear human to the human immune system, but rather the present result is more properly the idiotypic replacement of a non-human antibody with a fully human antibody which is generally unrelated structurally, but which binds to the same epitope on the same antigen, and preferably but not necessarily in the same orientation. Therefore, we propose referring to such antibodies as “idiologs”. The fact that in a single experiment a chimeric antibody Fab fragment could be converted to a series of semi-chimeric Fabs with human VH domains, all of which bind the same epitope on the same antigen with higher affinity than the starting chimeric Fab demonstrates the utility of the present invention for conversion of non-human antibodies to human idiologs with higher affinity for the same epitope, and by extension for affinity maturation of human antibodies as well.

1. An in vivo method for replacing a variable region in a reference antibody with a variable region from an antibody library, said method comprising the steps of:
a) ecombinantly altering a population of host cells by:
introducing into the host cells both a gene encoding a competitor having the binding properties of the reference antibody and a gene encoding a target molecule recognized by the competitor and reference antibody; and,
introducing into the host cells, members of a library of genes encoding hybrid antibodies, wherein the antibodies comprising the library have a variable region from the reference antibody and a variable region from an antibody library, so that cells of the population contain different hybrid antibodies; and,
b) etecting cells that both express the genes of step (a) and permit the binding of the hybrid antibody to the target in the presence of the competitor.

2. A method of claim 1 wherein the competitor is a reference antibody.

3. A method of claim 1 wherein the competitor is a natural ligand of the target molecule able to compete with the reference antibody for binding to the target molecule.

4. A method of claim 1 wherein the competitor is an artificial non-antibody ligand of the target molecule able to compete with the reference antibody for binding to the target binding partner.

5. A method of claim 1 wherein the competitor has a non-human Vh and a V1 region that is either human or non-human.

6. A method of claim 1 wherein the competitor is a hybrid antibody comprising human and nonhuman variable regions and the library encodes fully human antibodies.

7. A method of claim 1 where the competitor is a single chain antibody Fv fragment.

8. A method of claim 1 where the competitor is an antibody Fab fragment.

9. A method of claim 1 wherein the detection is by a self-inhibited reactivation system.

10. A method of claim 1 wherein the detection is by a competitive activation system.

11. A method of claim 1 where the test library members are bound to an activator or a detection system.

12. A method of claim 1 wherein the detection is by an enzyme fragment or subunit complementation system.

13. A method of claim 1 wherein a non-human variable region has been replaced by a human variable region.

14. A method of claim 1 wherein the selected antibody has a greater affinity for the target than does the reference antibody.

15. A method of claim 1 wherein the selected antibody is more stable than the reference antibody.

16. A method of claim 1 wherein the selected antibody has a greater specificity for the target than does the reference antibody.

17. A in vivo method for replacing a nonhuman antibody variable region with a human variable region in an antibody said method comprising the steps of:
a) ecombinantly altering a population of host cells by:
i. ntroducing into the host cells both a gene encoding a reference antibody having a nonhuman Vh or V1 and a gene encoding a target binding partner recognized by the reference antibody, and
ii. introducing into the host cells, members of a library of genes encoding antibody members have a variable region of the reference antibody and a human variable region from an antibody library so that cells of the population contain different hybrid binding partners, and;
b) detecting cells that both express the genes of step (a) and permit the binding of the hybrid binding partner to the target in the presence of the reference antibody.

18. A method of claim 17 wherein the reference antibody has a non-human Vh and a V1 region that is either human or non-human.

19. A method of claim 17 wherein the reference antibody comprises a human and nonhuman variable region and the library encodes fully human antibodies.

20. A method of claim 17 wherein the reference antibody comprises a non-human V1 and a human Vh.

21. A method of claim 17 where the reference antibody is a single chain antibody.

22. A method of claim 17 where the reference antibody is a Fab.

23. A method of claim 17 wherein the detection is by a self-inhibited reactivation system.

24. A method of claim 17 wherein the detection is by a competitive reactivation system.

25. A method of claim 17 where the library members are bound to an activator or a detection system.

26. A method of claim 17 wherein the detection is by a complementation system.

27. A method of claim 17 wherein the humanized antibody has a greater affinity for the target than the competitor binding pair partner.

28. A population of cells comprising:
i. recombinantly introduced gene encoding a competitor with the binding properties of a reference antibody;
ii. recombinantly introduced gene encoding a target molecule recognized by the competitor; and,
iii. library of genes encoding hybrid antibodies, wherein the antibodies comprising the library have a variable region from the reference antibody and a variable region from a natural antibody repertoire, so that cells of the population contain different hybrid antibodies.

29. A population of recombinantly altered cells of claim 28 comprising:
i. gene encoding a competitor that is a reference antibody having a nonhuman Vh or V1;
ii. gene encoding a target by the reference antibody; and,
ii. alibrary of genes encoding antibodies having a variable region of the reference antibody and a human variable region from an antibody library so that cells of the population contain different hybrid binding partners;

30. A population of claim 28 wherein the library members are attached to a part of multipart reporter system.

31. A population of claim 28 wherein the target molecule has a self-inhibited reporter attached to it.
